(19) United States
(12) Patent Application Publication
(10) Pub. No.: US 2024/0379243 A1
(43) Pub. Date: Nov. 14, 2024

(54) MACHINE LEARNING CARDIOVASCULAR CONDITION PROGRESSION

(71) Applicant: Oxford University Innovation Limited, Oxford (GB)

(72) Inventors: Paul LEESON, Oxford (Oxfordshire) (GB); Winok LAPIDAIRE, Oxford (Oxfordshire) (GB); Maryam ALSHARQI, Oxford (Oxfordshire) (GB); Andrew FLETCHER, Oxford (Oxfordshire) (GB); Adam LEWANDOWSKI, Oxford (Oxfordshire) (GB)

(21) Appl. No.: 18/691,252

(22) PCT Filed: Sep. 16, 2022
(86) PCT No.: PCT/GB2022/052353

§ 371 Date: Mar. 12, 2024

(30) Foreign Application Priority Data
Sep. 17, 2021 (GB) 2113322.8
Aug. 25, 2022 (GB) 2212362.4

(51) Int. Cl. G16H 50/50; G16H 50/70

(52) U.S. Cl. CPC G16H 50/50; G16H 50/70

(57) ABSTRACT

A method calculates a score representing cardiovascular condition progression cardiovascular condition using feature data comprising cardiovascular image features from a population including a background group and a target group at a later stage of the condition. Contrastive principal component analysis is applied between feature data from the background and target groups to obtain a transformation into a reduced representation space, which is applied to the population feature data to determine positions of each individual in the space. Trajectories are determined in the space between the target and background groups by connecting the positions. The score is calculated as a distance along the one of the trajectories on which the position of an individual lies. Another method calculates a contribution of each of the plurality of features to the transformation, and determines a plurality of features having the highest contributions. Other methods derive models for calculating the score by fitting.

The invention relates to methods for using machine-learning to analyse data about progression of a cardiovascular condition of interest using contrastive principal component analysis.

Hypertension in young adults is associated with an increased risk of early stroke and cardiovascular disease [1, 2]. Early identification of subclinical alterations may prevent or delay the onset of adverse events [3, 4]. However, hypertension management in young adult is challenging due to the lack of longitudinal assessment of the progression of the underlying disease within different organs. Due to lack of sufficient data about the risk stratification strategies for patients below the age of 40, hypertension management in young patients is based on considerable extrapolation [5-7]. Current data on the management of hypertension and prevention of cardiovascular disease have been established from populations over 40 years of age. However, hypertensives below the age of 40 are known to have different pathophysiological responses to high blood pressure [8, 9]. In younger patients, there is a lack of longitudinal datasets with a sufficient follow-up duration to assess the long-term treatment effect and detection of signs of target organ damage [6].

Cross-sectional datasets are limited to a single snapshot of parameters for each subject, which therefore limit the ability to study the disease progression later in life without the availability of follow-up data. In cross-sectional early assessments of hypertension, left atrial deformation indices have been reported as independent predictors of adverse events in patients with hypertension and heart failure [10, 11]. However, using singular variables in prediction models has given inconsistent results across populations [12].

Machine learning tools offer the integration of multi-dimensional phenotypes and identify particular disease patterns [13]. In cancer genomics, researchers have been using unsupervised machine learning techniques that extract temporal information from cross-sectional datasets to order subjects based on the severity of the disease [14]. The extracted pseudo-temporal data allowed mapping of the dynamic biological and pathological mechanisms over the course of disease from cross-sectional datasets [14-16]. Iturria-Medina et al. revealed temporal patterns of a neurodegenerative population by integrating cross-sectional gene expression data. This algorithm generated a score to order patients with Alzheimer's diseases relative to a comparison healthy population. The scores predicted the neuropathological severity and clinical deterioration to advanced disease stages [17].

It would be desirable to provide an algorithm that is capable of obtaining similar results for cardiovascular conditions, including diseases such as hypertension and related conditions such as diastolic heart failure, where cross-sectional multi-dimensional datasets are available, but longitudinal datasets are lacking.

According to a first aspect of the invention, there is provided a method of calculating a score representative of a progression of a cardiovascular condition, wherein the method is performed on feature data from individuals in a population including a background group of individuals and a target group of individuals at a later stage of the cardiovascular condition than the background group of individuals, the feature data comprising a plurality of features for each individual including a plurality of cardiovascular image features, and the method comprises: applying contrastive principal component analysis between feature data from the background group of individuals and feature data from the target group of individuals to obtain a transformation into a reduced representation space; applying the transformation to the feature data from the population of individuals to determine a position of each individual of the population in the reduced representation space; determining trajectories in the reduced representation space between the target group and the background group by connecting the positions of the individuals of the population in the reduced representation space; and for one or more of the individuals of the population, calculating the score as a distance along the one of the trajectories on which the position of the individual lies.

By using contrastive principal component analysis, trajectories can be obtained using cross-sectional data that represent progression of a cardiovascular condition. This in turn can be used to determine a score for an individual without having to track each individual's state over time.

In some embodiments, the method further comprises calculating a contribution of each of the plurality of features to the transformation, and determining a plurality of the features having the highest contributions to the transformations. Determining the features that contribute most to the transformation allows for the identification of which features are most significant for assessing the progression of the condition of interest. This in turn can simplify and speed up future assessments for other individuals.

In some embodiments, the population further includes a reference group of individuals at a stage of the cardiovascular condition intermediate the background group of individuals and the target group of individuals. Performing the contrastive principal component analysis on only a subset of the data improves efficiency, and can further improve contrast particularly when combined with careful choice of the target and background groups.

In some embodiments, the population of individuals further includes at least one test subject at an unknown stage of the cardiovascular condition, and the one or more individuals for whom the score is calculated comprises the at least one test subject. The method can be used to determine a score for new test subjects by including the subjects alongside the reference individuals making up the original population dataset.

In some embodiments, the method further comprises calculating a matrix of distances among the positions of the individuals of the population in the reduced representation space, the step of determining trajectories being performed on the basis of the matrix of distances. This matrix allows the method to assess the spatial relationships between the positions in order to determine how to connect them into trajectories.

In some embodiments, the step of determining trajectories comprises: determining a minimum spanning tree among the positions of the population in the reduced representation space based on the matrix of distances; and defining the trajectories as paths within the minimum spanning tree. The minimum spanning tree is a convenient and efficient algorithm for connecting all of the positions to form trajectories that connect neighbouring positions representing similar disease states.

In some embodiments, the distances are Euclidean distances. A Euclidean distance is a well-established way to evaluate the distances between points in a multi-dimensional space.

In some embodiments, the step of determining trajectories further comprises identifying one or more subtrajectories representing paths in the reduced representation space based on the matrix of distances, each subtrajectory comprising a plurality of the trajectories, and assigning each individual of the population to one or more of the subtrajectories. By identifying subtrajectories comprising plural similar trajectories of individuals, it is possible to identify common paths of disease progression.

In some embodiments, the identifying of the one or more subtrajectories comprises performing spectral clustering over the matrix of distances. Spectral clustering is a well-understood method for grouping together similar elements and provides a convenient method to form subtrajectories.

In some embodiments, the trajectories connect to a reference point and the distance along the one of the trajectories on which the position of the individual lies is a distance between the position of the individual and the reference point. Using a reference allows all of the trajectories to have a consistent endpoint, so that the scores are more comparable between trajectories.

In some embodiment, the reference point is an average position in the reduced representation space of individuals in the background group. This choice of reference point means that the score provides a measure of the severity of the condition, with a larger score indicating more severe progression of the condition.

According to a second aspect of the invention, there is provided a method of analysing feature data about a cardiovascular condition wherein the method is performed on feature data from individuals in a population including a background group of individuals and a target group of individuals at a later stage of the cardiovascular condition than the background group of individuals, the feature data comprising a plurality of features for each individual including a plurality of cardiovascular image features, and the method comprises: applying contrastive principal component analysis between feature data from the background group of individuals and feature data from the target group of individuals to obtain a transformation into a reduced representation space; calculating a contribution of each of the plurality of features to the transformation; and determining a plurality of the features having the highest contributions to the transformation.

Determining the features that contribute most to the transformation allows for the identification of which features are most significant for assessing the progression of the condition of interest. This in turn can simplify and speed up future assessments for other individuals.

In some embodiments, the method further comprises: applying the transformation to the feature data from the population of individuals to determine a position of each individual of the population in the reduced representation space; determining trajectories in the reduced representation space between the target group and the background group by connecting the positions of the individuals of the population in the reduced representation space; and for one or more of the individuals of the population, calculating the score as a distance along the one of the trajectories on which the position of the individual lies. By using the transformation obtained from the contrastive principal component analysis, trajectories can be obtained using cross-sectional data that represent progression of a cardiovascular condition. This in turn can be used to determine a score for an individual without having to track each individual's state over time.

In some embodiments, calculating the contribution comprises, for one or more principal components from the contrastive principal component analysis, calculating a product of an eigenvalue of the principal component with a loading of the feature for the principal component; and the contribution for the feature comprises a sum of the products. By calculating the sum of these products, an overall significance of the feature to the transformation can be estimated.

In some embodiments, the one or more principal components comprises principal components having an eigenvalue above a predetermined value. Excluding principal components with low significance simplifies the calculation, particularly in the case where there are a large number of principal components.

In some embodiments, the product is normalised by a sum for the principal component of the loadings of the plurality of features. This normalisation improves the comparability of the values, as the loadings for each principal component may not sum to the same value.

In some embodiments, the method further comprises a step of pre-processing the feature data to obtain processed feature data, wherein the steps of applying contrastive principal component analysis and applying the transformation are performed using the processed feature data. Pre-processing the feature data can be used to ensure that the data is consistent and of sufficient quality to permit further analysis.

In some embodiments, pre-processing the feature data comprises adjusting the feature data to account for one or more confounding factors. In some embodiments, the confounding factors comprise one or more of a sex of each of the individuals, an age of each of the individuals, a condition under which the feature data was measured, and a medication regime of each of the individuals. This is advantageous where feature data is derived from multiple sources, and different procedures or conditions may affect the data.

In some embodiments, pre-processing the feature data comprises imputing missing values for one or more of the features for one or more of the individuals. This can allow data to still be used where it is incomplete.

In some embodiments, pre-processing the feature data comprises selecting a subset of the features based on a comparison for each feature of a local variance of the feature with a global variance of the feature. This allows the method to prefer features that vary in a manner that is indicative of a smooth progression through the reduced representation space.

In some embodiments, the step of applying contrastive principal component analysis comprises applying a contrast parameter to the feature data from the background group. This allows the contrastive principal component analysis to be optimised between having a high target variance and a low background variance.

In some embodiments, the step of applying contrastive principal component analysis comprises applying the contrastive principal component analysis a plurality of times using different values of the contrast parameter to obtain a plurality of different transformations, and selecting one of the plurality of transformations, wherein the step of applying the transformation uses the selected transformation. By using a range of values of the contrast parameter, the method can choose values that provide improved contrast in the reduced representation space.

In some embodiments, selecting one of the plurality of transformations comprises automatically selecting one of the plurality of transformations. Automatic selection is advantageous because it can be performed more quickly and consistently than manual selection, thereby improving the efficiency and consistency of the method.

In some embodiments, automatically selecting one of the plurality of transformations comprises: for each of the plurality of transformations: determining positions of each of the individuals of the population in a reduced representation space using the transformation; assigning each position to one of a plurality of clusters in the reduced representation space; and calculating a clustering parameter using the positions, the clustering parameter comparing a dispersion within each of the clusters to a reference distribution; selecting a transformation from the plurality of transformations based on the clustering parameter. This prefers values that cause the trajectories to cluster, thereby improving the ability to resolve distinct paths of progression of the condition through the reduced representation space.

In some embodiments, applying contrastive principal component analysis comprises applying kernel contrastive principal component analysis, such that the transformation into the reduced representation space is non-linear. Non-linear transformations provide greater flexibility in the nature of the transformation. Although they are more complex, this can potentially provide further optimisation of the transformation for resolving progression of the condition.

According to a third aspect of the invention, there is provided a method of determining a subject score representative of a progression of a cardiovascular condition for a test subject comprising: determining a position of the test subject in a reduced representation space by applying a transformation into the reduced representation space obtained using the method of any one of the preceding aspects to subject feature data from the test subject, the subject feature data comprising data on a plurality of features for the test subject including a plurality of cardiovascular image features; determining a position of the test subject on one of a plurality of trajectories in the reduced representation space determined using the method of claim 1 or any claim dependent thereon; and calculating the subject score using a position along the one of the trajectories on which the position of the subject lies.

By using a predetermined transformation and predetermined trajectories, a score can be calculated for a new subject without having to repeat the calculations that determine the transformation and the trajectories. This allows scores to be calculated for new subject more efficiently and conveniently.

In some embodiments, the plurality of features is a plurality of features having the highest contributions to the transformation determined using a method according to the second aspect. By only using the most significant features identified, an accurate score can be determined while reducing the amount of data that must be gathered for new subjects.

The following comments apply to all aspects of the present invention.

In some embodiments, the plurality of cardiovascular image features are determined from echocardiogram images. Echocardiogram images are safe and widely used for assessing cardiovascular condition states, so are a valuable source of feature data.

In some embodiments, the plurality of cardiovascular image features are determined from cardiac images. Other types of cardiac imaging can provide valuable information about the heart that can aid in diagnosis of other cardiac conditions.

In some embodiments, the method further comprises a step of determining the cardiovascular image features from images from each of the respective individuals. In some cases, it may be necessary to extract the appropriate feature data from the raw echocardiogram images.

In some embodiments, the feature data further comprises clinical data about each of the respective individuals, the clinical data comprising one or more of: an age of the individual, a sex of the individual, an ethnicity of the individual, a height of the individual, a weight of the individual, and a medication regime of the individual. Including further clinical and contextual data about the subjects can improve the accuracy of the method.

In some embodiments, the condition of interest is a disease such as hypertension or associated cardiac conditions such as diastolic dysfunction. Hypertension is a desirable target for cross-sectional analysis, particularly for younger subjects where longitudinal data over a long period of time is not readily available.

According to a fourth aspect of the invention, there is provided a method of calculating a subject score representative of a progression of a cardiovascular condition for a test subject, wherein the method is performed on reference feature data from individuals in a population including a background group of individuals and a target group of individuals at a later stage of the cardiovascular condition than the background group of individuals, the reference feature data comprising a plurality of features for each individual including a plurality of cardiovascular image features, and the method comprises: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; for each individual of the population, calculating a reference score representative of the progression of the cardiovascular condition as a distance along the one of the trajectories on which the position of the individual lies; performing fitting between the reference feature data and the reference scores to derive a model for calculating a score representative of the progression of the cardiovascular condition, wherein the model uses a subset of two or more of the plurality of features to calculate the score; and applying the model to subject feature data from the test subject to obtain the subject score, the subject feature data comprising data on the subset of features for the test subject.

This model allows a simple model to be obtained that produces comparable results to the complete model based on the theoretically-justified process of determining trajectories. However, the simple model has the advantage of being less computationally expensive and potentially requiring data on fewer features.

In some embodiments, the fitting comprises selecting the subset of one or more of the plurality of features, optionally wherein the subset comprises fewer than all of the plurality of features. The choice of which features are used can affect the performance of the simple model, and so it is advantageous to select particular subsets. Using fewer features reduces the computational load and makes obtaining sufficient data easier.

In some embodiments, the subset is selected based on an accuracy of the model using the subset of features. Choosing the features that provide the most accurate simple model ensures the results of the simple model are as close to those of the full model as possible.

In some embodiments, the subset is selected based on an ease of obtaining subject feature data comprising data on the subset of features. In some cases it may desirable to select features for which data is easily obtained or more readily available, even if this may come at the expense of slightly reduced accuracy in some situations.

In some embodiments, the fitting comprises regression analysis. In some embodiments, the regression analysis comprises linear regression. These are readily calculated analysis techniques that are suitable to the present application and can be implemented in a convenient and efficient manner.

In some embodiments, selecting the subset comprises using stepwise regression analysis. This allows multiple combinations of features to be automatically assessed for suitability, for example according to the criteria mentioned above.

In some embodiments, the population further includes a reference group of individuals at a stage of the cardiovascular condition intermediate the background group of individuals and the target group of individuals. Performing the contrastive principal component analysis on only a subset of the data improves efficiency, and can further improve contrast particularly when combined with careful choice of the target and background groups.

In some embodiments, the method further comprises: calculating a matrix of distances among the positions of the individuals of the population in the reduced representation space, the step of determining trajectories being performed on the basis of the matrix of distances. This matrix allows the method to assess the spatial relationships between the positions in order to determine how to connect them into trajectories.

In some embodiments, the step of determining trajectories comprises: determining a minimum spanning tree among the positions of the population in the reduced representation space based on the matrix of distances; and defining the trajectories as paths within the minimum spanning tree. The minimum spanning tree is a convenient and efficient algorithm for connecting all of the positions to form trajectories that connect neighbouring positions representing similar disease states.

In some embodiments, the distances are Euclidean distances. A Euclidean distance is a well-established way to evaluate the distances between points in a multi-dimensional space.

In some embodiments, the trajectories connect to a reference point and the distance along the one of the trajectories on which the position of the individual lies is a distance between the position of the individual and the reference point. Using a reference allows all of the trajectories to have a consistent endpoint, so that the scores are more comparable between trajectories.

In some embodiments, the reference point is an average position in the reduced representation space of individuals in the background group. This choice of reference point means that the score provides a measure of the severity of the condition, with a larger score indicating more severe progression of the condition.

In some embodiments, the method further comprises a step of pre-processing the feature data to obtain processed feature data, wherein the steps of applying contrastive principal component analysis and applying the transformation are performed using the processed feature data. Pre-processing the feature data can be used to ensure that the data is consistent and of sufficient quality to permit further analysis.

In some embodiments, pre-processing the feature data comprises adjusting the feature data to account for one or more confounding factors. In some embodiments, the confounding factors comprise one or more of a sex of each of the individuals, an age of each of the individuals, a condition under which the feature data was measured, and a medication regime of each of the individuals. This is advantageous where feature data is derived from multiple sources, and different procedures or conditions may affect the data.

In some embodiments, pre-processing the feature data comprises imputing missing values for one or more of the features for one or more of the individuals. This can allow data to still be used where it is incomplete.

In some embodiments, pre-processing the feature data comprises selecting a subset of the features based on a comparison for each feature of a local variance of the feature with a global variance of the feature. This allows the method to prefer features that vary in a manner that is indicative of a smooth progression through the reduced representation space.

In some embodiments, the step of applying contrastive principal component analysis comprises applying a contrast parameter to the feature data from the background group. This allows the contrastive principal component analysis to be optimised between having a high target variance and a low background variance.

In some embodiments, the step of applying contrastive principal component analysis comprises applying the contrastive principal component analysis a plurality of times using different values of the contrast parameter to obtain a plurality of different transformations, and selecting one of the plurality of transformations, wherein the step of applying the transformation uses the selected transformation. By using a range of values of the contrast parameter, the method can choose values that provide improved contrast in the reduced representation space.

In some embodiments selecting one of the plurality of transformations comprises automatically selecting one of the plurality of transformations. Automatic selection is advantageous because it can be performed more quickly and consistently than manual selection, thereby improving the efficiency and consistency of the method.

In some embodiments, automatically selecting one of the plurality of transformations comprises: for each of the plurality of transformations: determining positions of each of the individuals of the population in a reduced representation space using the transformation; assigning each position to one of a plurality of clusters in the reduced representation space; and calculating a clustering parameter using the positions, the clustering parameter comparing a dispersion within each of the clusters to a reference distribution; selecting a transformation from the plurality of transformations based on the clustering parameter. This prefers values that cause the trajectories to cluster, thereby improving the ability to resolve distinct paths of progression of the condition through the reduced representation space.

In some embodiments, applying contrastive principal component analysis comprises applying kernel contrastive principal component analysis, such that the transformation into the reduced representation space is non-linear. Non-linear transformations provide greater flexibility in the nature of the transformation. Although they are more complex, this can potentially provide further optimisation of the transformation for resolving progression of the condition.

In some embodiments, the plurality of cardiovascular image features are determined from echocardiogram images. Echocardiogram images are safe and widely used for assessing cardiovascular condition states, so are a valuable source of feature data.

In some embodiments, the plurality of cardiovascular image features are determined from cardiac images. Other types of cardiac imaging can provide valuable information about the heart that can aid in diagnosis of other cardiac conditions.

In some embodiments, the method further comprises a step of determining the cardiovascular image features from images from each of the respective individuals. In some cases, it may be necessary to extract the appropriate feature data from the raw echocardiogram images.

In some embodiments, the feature data further comprises clinical data about each of the respective individuals, the clinical data comprising one or more of: an age of the individual, a sex of the individual, an ethnicity of the individual, a height of the individual, a weight of the individual, and a medication regime of the individual. Including further clinical and contextual data about the subjects can improve the accuracy of the method.

In some embodiments, the cardiovascular condition is hypertension, cardiac disease, or diastolic dysfunction. Hypertension is a desirable target for cross-sectional analysis, particularly for younger subjects where longitudinal data over a long period of time is not readily available.

According to a fifth aspect of the invention, there is provided a method of calculating a subject score representative of a progression of a cardiovascular condition for a test subject, wherein the method is performed on reference feature data and reference scores representative of the progression of the cardiovascular condition; the reference feature data is from individuals in a population including a background group of individuals and a target group of individuals at a later stage of the cardiovascular condition than the background group of individuals, the reference feature data comprising a plurality of features for each individual including a plurality of cardiovascular image features; the reference scores are obtained by: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; and for each individual of the population, calculating the reference score as a distance along the one of the trajectories on which the position of the individual lies; and the method comprises: performing fitting between the reference feature data and the reference scores to derive a model for calculating a score representative of the progression of the cardiovascular condition, wherein the model uses a subset of one or more of the plurality of features to calculate the score; and applying the model to subject feature data from the test subject to obtain the subject score, the subject feature data comprising data on the subset of features for the test subject. Determining the model based on previously-determined trajectory data means that the relatively computationally-expensive process of determining trajectories does not need to be performed at the time of deriving the simplified model.

According to a sixth aspect of the invention, there is provided a method of determining a subject score representative of a progression of a cardiovascular condition for a test subject, wherein: the method uses a model for calculating a score representative of the progression of the cardiovascular condition; the model uses a set of one or more features to calculate the score; the model is derived using reference feature data from individuals in a population including a background group of individuals and a target group of individuals at a later stage of the cardiovascular condition than the background group of individuals, the reference feature data comprising a plurality of features for each individual including a plurality of cardiovascular image features, and the model is derived by: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; for each individual of the population, calculating a reference score representative of the progression of the cardiovascular condition as a distance along the one of the trajectories on which the position of the individual lies; performing fitting between the reference feature data and the reference scores to derive the model for calculating a score representative of the progression of the cardiovascular condition using the set of one or more features, wherein the set of one or more features is a subset of the plurality of features; the method comprises: applying the model for calculating a score representative of the progression of the cardiovascular condition to subject feature data from the test subject to obtain the subject score, the subject feature data comprising data on the set of features for the test subject. Applying a previously-derived simplified model further reduces the computational burden at the point of use.

According to further aspects of the invention, there are provided computer programs comprising instructions which, when the program is executed by a computer, cause the computer to carry out the method of any of the preceding aspects. There are further provided systems comprising a processor configured to carry out the method of any of the preceding aspects.

Embodiments of the present invention will now be described by way of non-limitative example with reference to the accompanying drawings, in which:

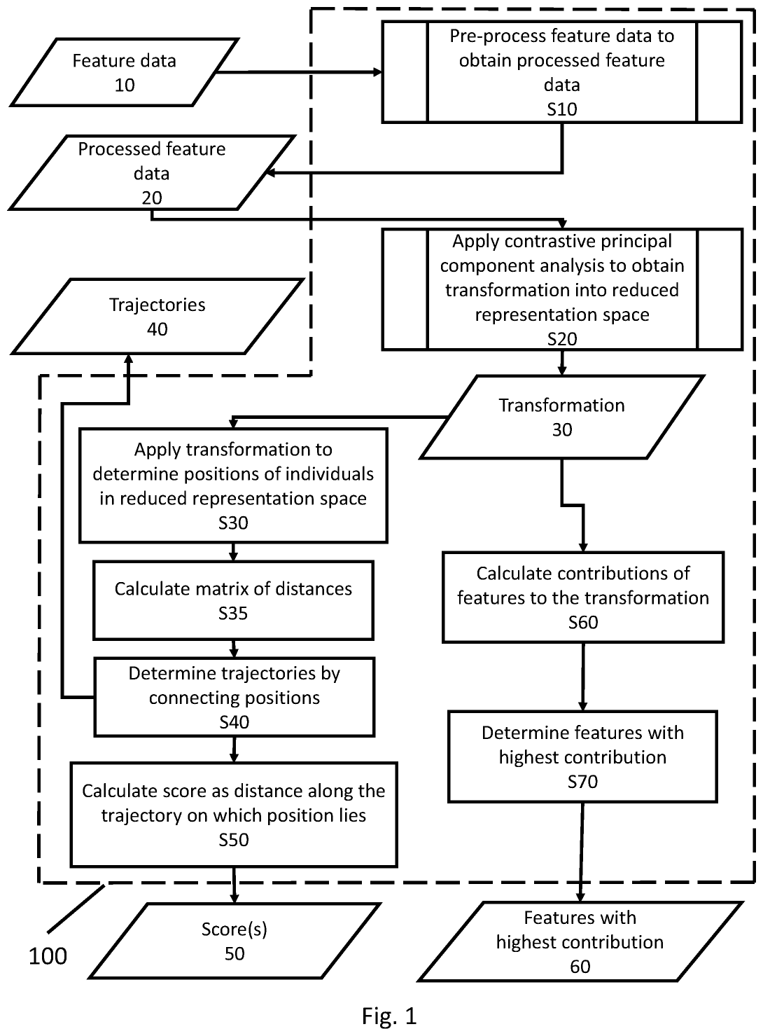

FIG. 1 is a flowchart showing the methods of the first and second aspects of the invention;

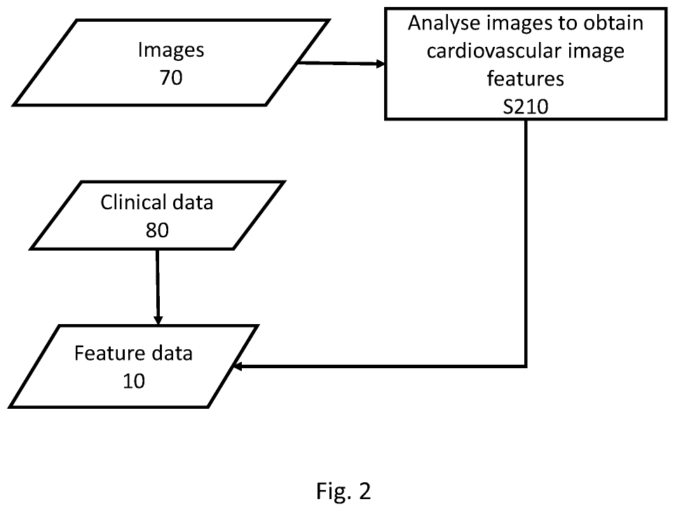

FIG. 2 is a flowchart showing how the feature data may be derived;

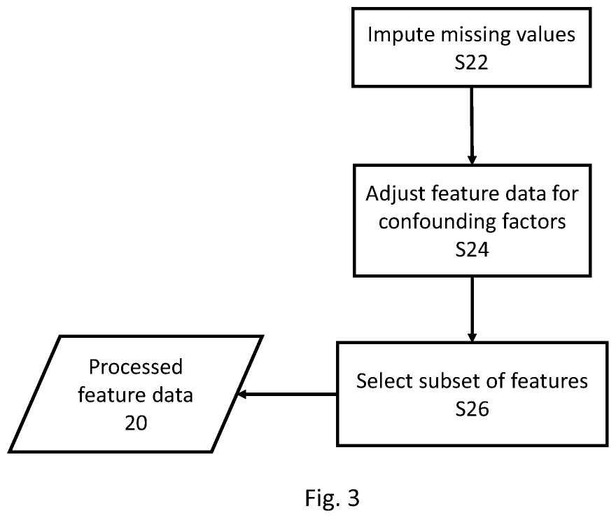

FIG. 3 is a flowchart showing further detail of the pre-processing of the feature data;

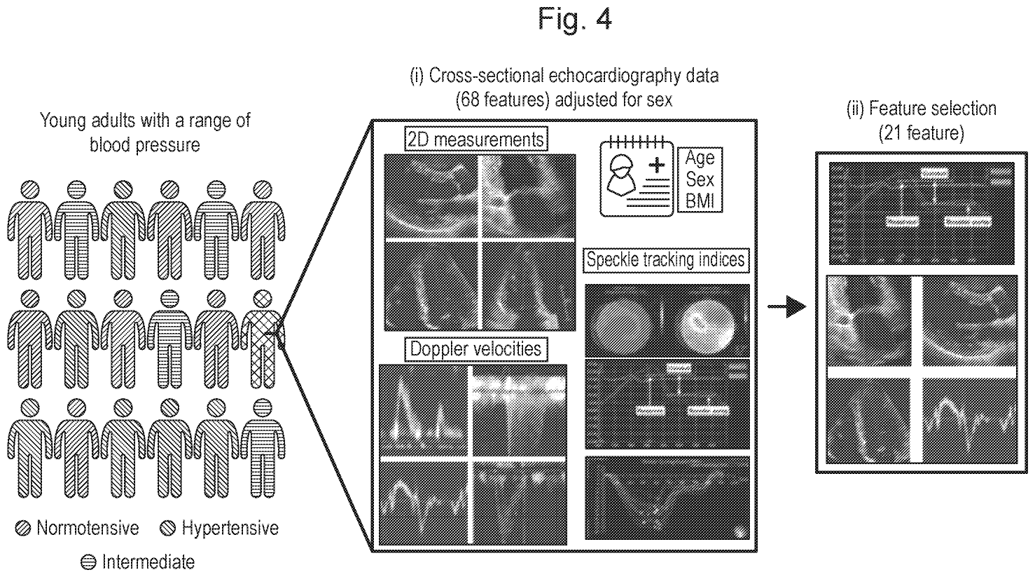

FIG. 4 shows examples of echocardiogram images and feature data;

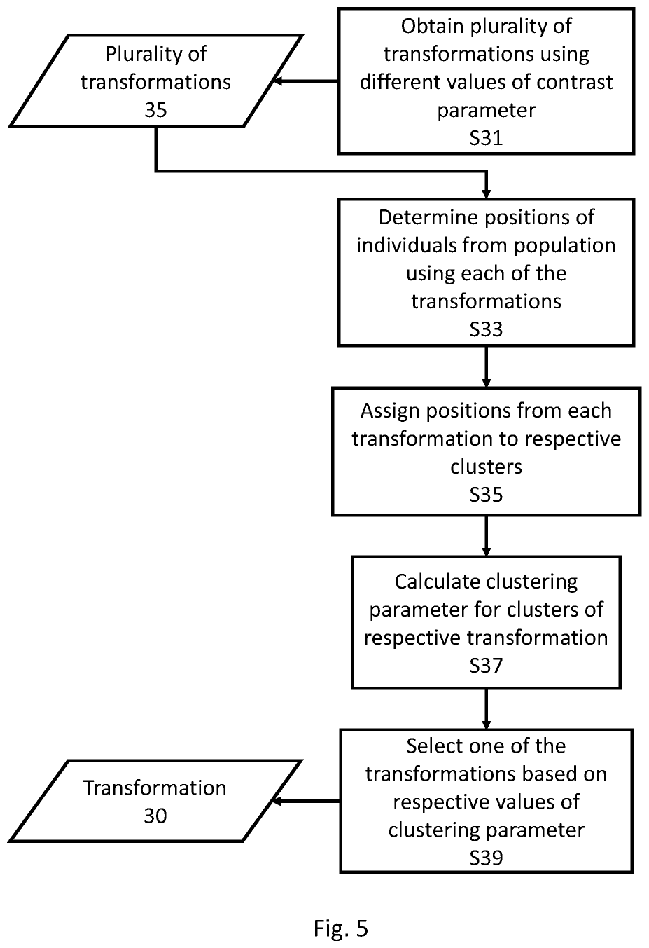

FIG. 5 is a flowchart showing further detail of the determination of the transformation into the reduced representation space;

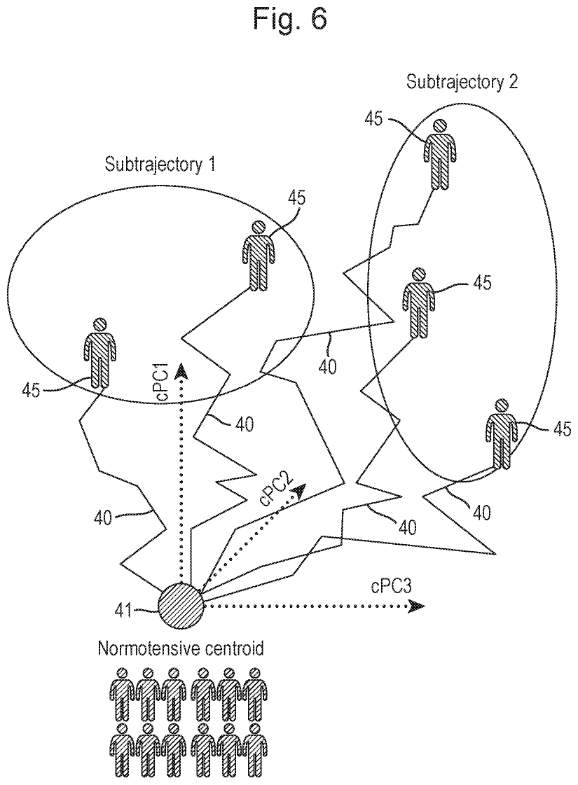

FIG. 6 shows illustrative trajectories in the reduced representation space;

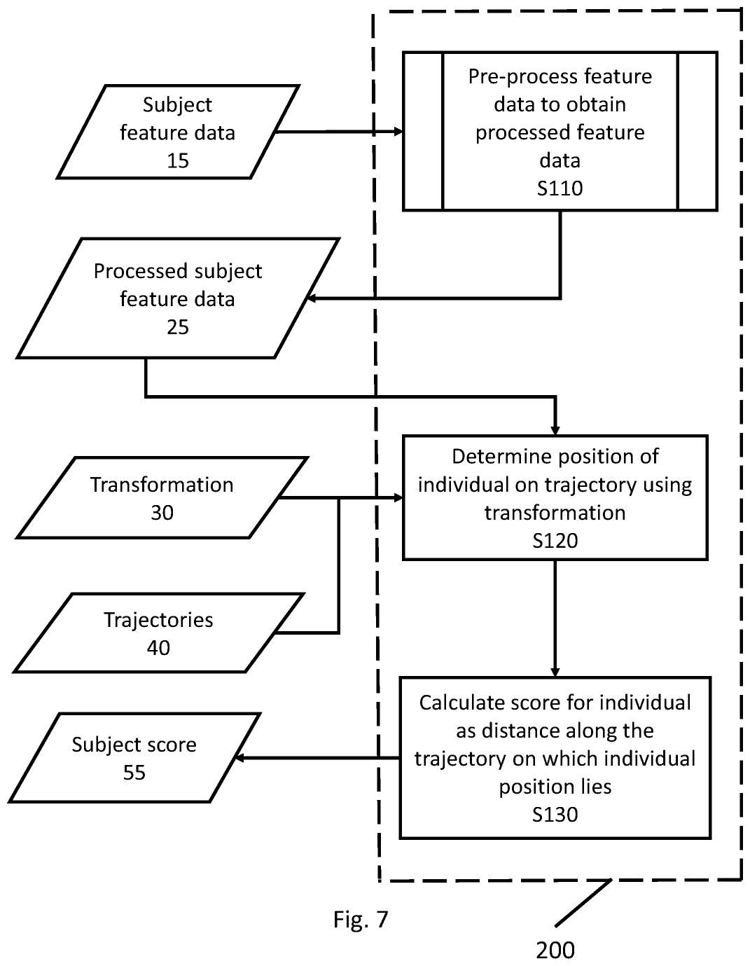

FIG. 7 is a flowchart showing the method of the third aspect of the invention;

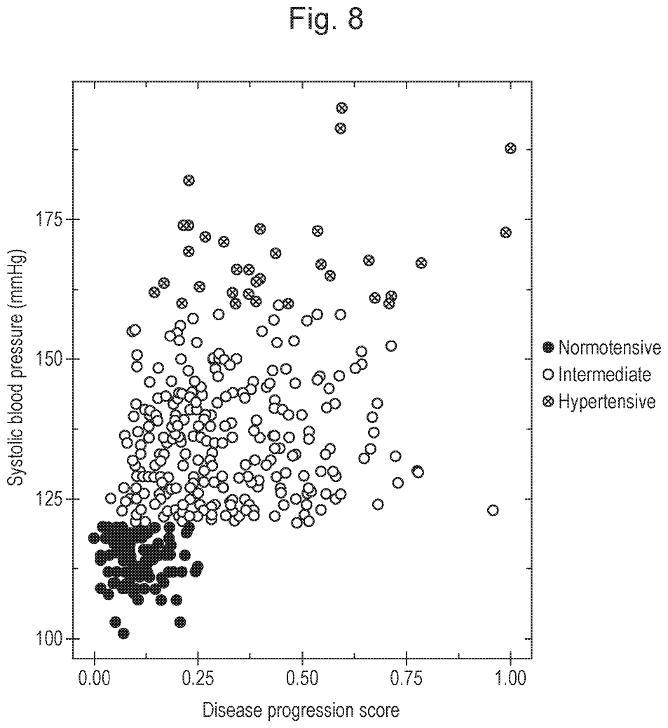

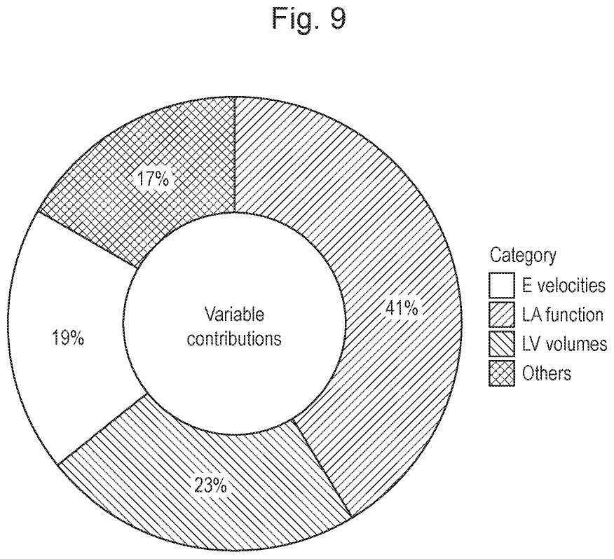

FIG. 8 shows data illustrating the relationship between blood pressure and disease score in an embodiment where the method is applied to determine a disease score for hypertension;

FIG. 9 shows the contribution to the transformation from different categories of feature in an embodiment where the method is applied to determine a disease score for hypertension;

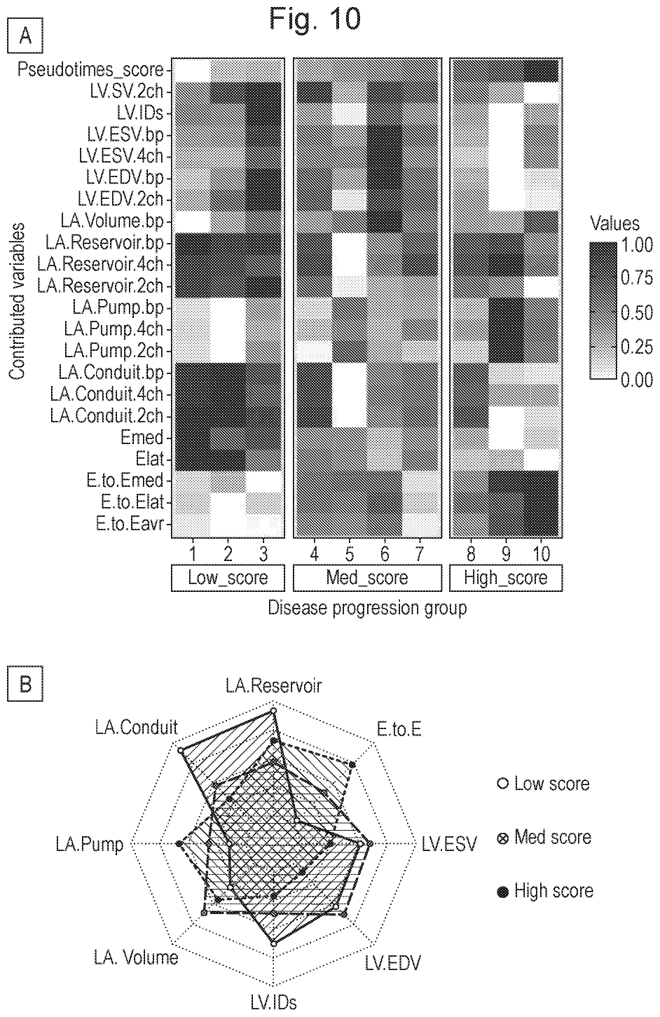

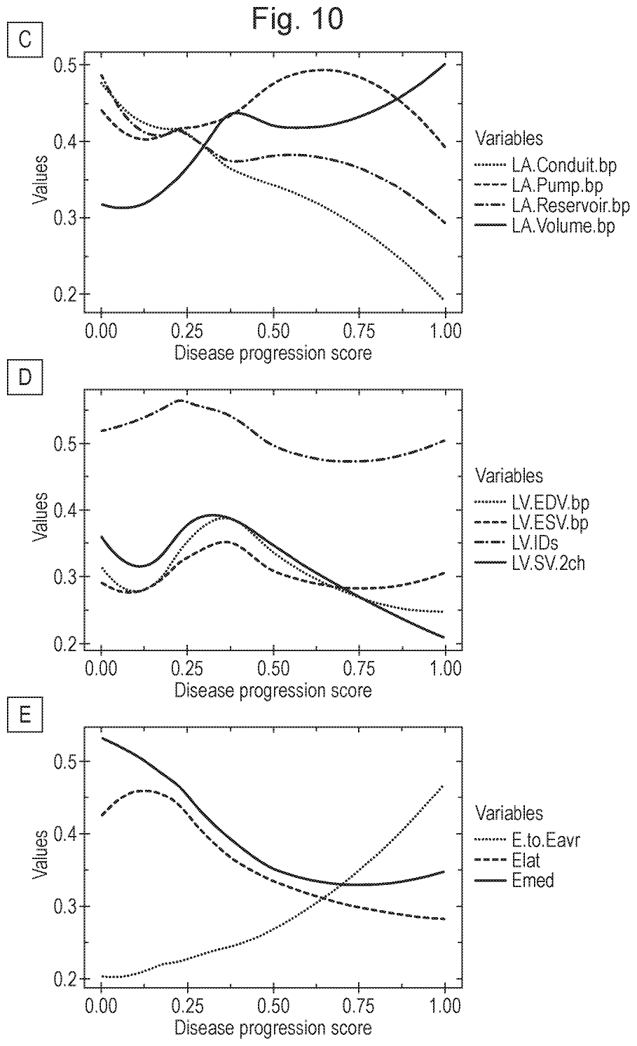

FIG. 10 shows correlation between the disease progression score and features in the feature data in an embodiment where the method is applied to determine a disease score for hypertension;

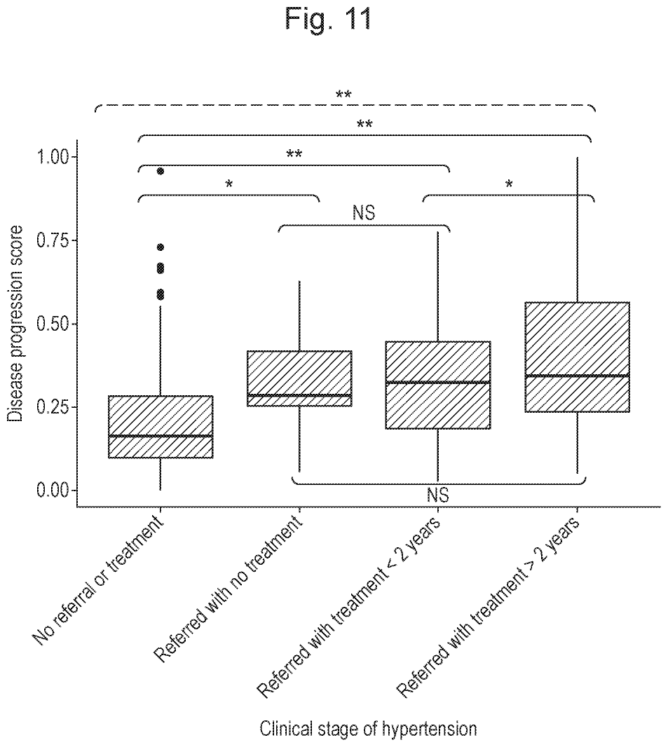

FIG. 11 shows correlation between the disease progression score and clinical interventions for corresponding individuals in an embodiment where the method is applied to determine a disease score for hypertension;

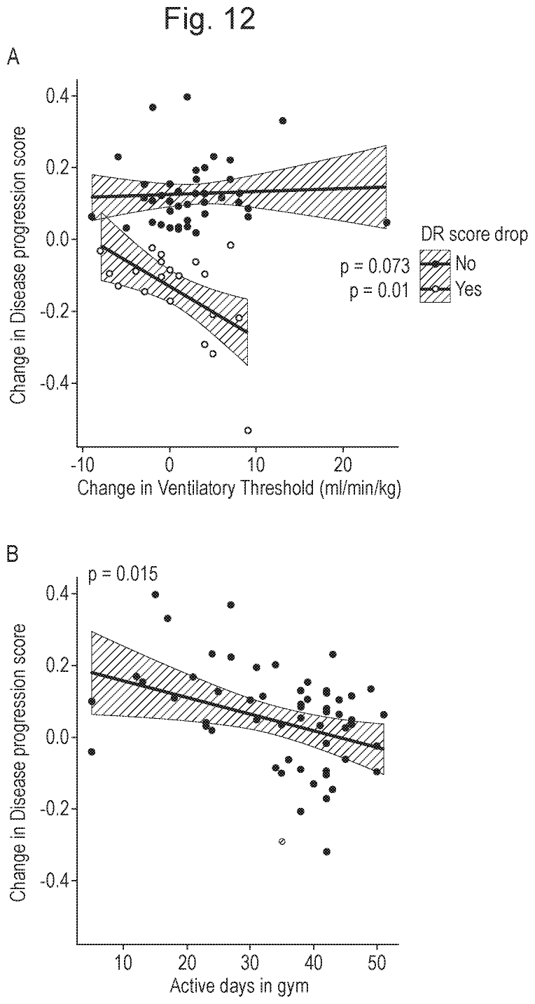

FIG. 12 shows the effect of an exercise programs on the disease score for individuals in an embodiment where the method is applied to determine a disease score for hypertension;

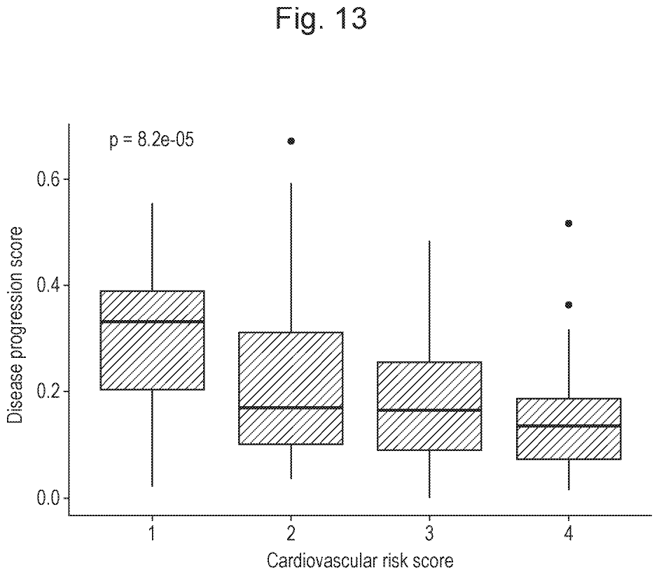

FIG. 13 shows correlation between the disease progression score and the cardiovascular risk score in an embodiment where the method is applied to determine a disease score for hypertension;

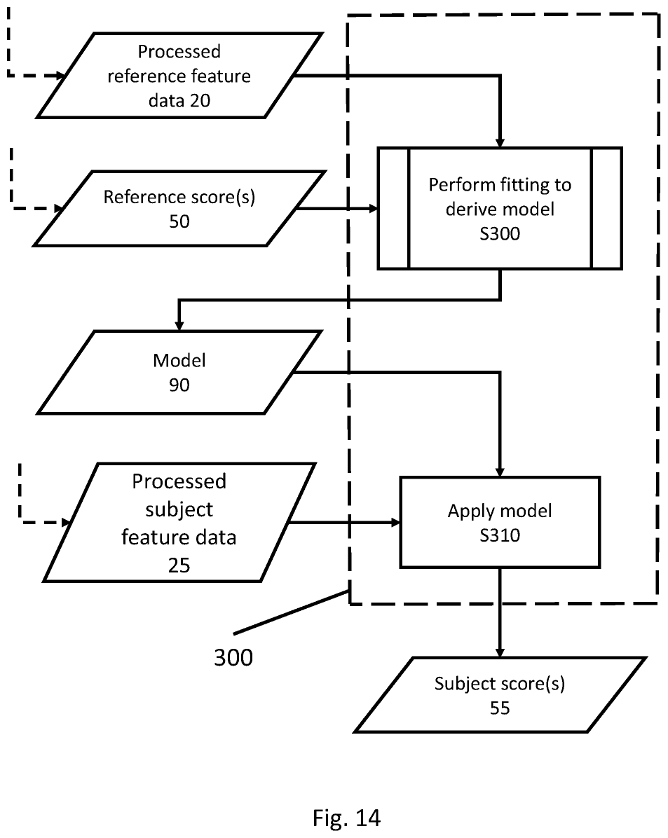

FIG. 14 is a flowchart illustrating an alternative method for calculating a disease progression score according to the fourth, fifth, and sixth aspects; and

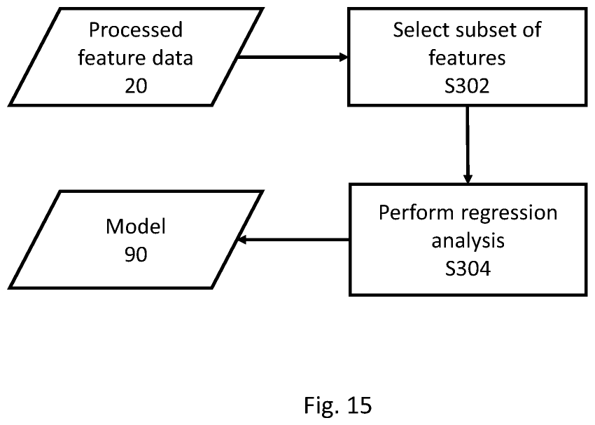

FIG. 15 is a flowchart showing further detail of the step of performing fitting to derive a model.

FIG. 1 is a flowchart of a method of calculating a score 50 representative of a progression of a cardiovascular condition. For example, the cardiovascular condition may be a cardiovascular disease such as hypertension, cardiac disease, or diastolic dysfunction. As discussed above, the method allows for cross-sectional data on cardiovascular conditions to be used to assess progression of the condition, rather than longitudinal data sets that require tracking individuals over time. Where the cardiovascular condition is a cardiovascular disease, the score 50 may be referred to as a disease score.

The method is performed on feature data 10 from individuals in a population. The population includes a background group of individuals and a target group of individuals at a later stage of the cardiovascular condition than the background group of individuals. The background and target groups may be manually selected, for example based on clinical or medical data such as a diagnosis or assessment from a medical professional. To define the background group, a user may provide a list of IDs, for example identifying the individuals from a database or list containing the entire population.

In some embodiments, all the other individuals in the population not defined as part of the background group are taken as the target group. In such embodiments, only the background group needs to be explicitly defined. The inverse may also be used, i.e. that only the target group is explicitly defined and all other individuals in the population are taken as the background group. The exact method by which the background and target groups are defined can vary as long as the individuals in the target group are at a later stage of the cardiovascular condition than the background group of individuals. This may be assessed, for example, by an average progression of the condition among individuals in the target group versus an average progression of the condition among individuals in the background group.

In some embodiments, the user may be interested in defining both the target group and the background group with particular subsets of individuals from the population (e.g. individuals notably late and early in the condition progression respectively). In this case, the population further includes a reference group of individuals at a stage of the cardiovascular condition intermediate the background group of individuals and the target group of individuals. This may be advantageous for improving the contrast between the target group and the background group when applying the contrastive methods described in more detail below.

The choice of the background group and the target group can have a strong influence on the output of the method [18]. It is advantageous if the choice takes into account the cardiovascular condition. In particular, the background group may comprise individuals not having the cardiovascular condition. The target group may comprise individuals having the cardiovascular condition. In addition, it can be advantageous if the background group is chosen to have similar demographic characteristics to the target group. This further ensures that the differences between the target group and background group are more likely to be due to the cardiovascular condition. The target group may comprise an heterogeneous population, but, if a subset of individuals with highly similar pathological stages/variants is considerably more abundant than subjects at other stages/variants, this subset could statistically dominate (and bias) the contrastive principal component analysis technique discussed in more detail below. In such cases, it is preferred if the target group is defined as a group of individuals having an equilibrated compendium of disease stages/variants.

For example, in the embodiments below which concern determining scores for hypertension, resting blood pressure measurements were used to categorise the individuals in the population into three groups:
Hypertensive (individuals with systolic blood pressure ≥160 mmHg);
Normotensive (individuals with systolic blood pressure <120 mmHg, and not on antihypertension medication); and
Intermediate (individuals with systolic blood pressure ≥120 mmHg and <160 mmHg).

The hypertensive individuals were defined as the target group, the normotensive individuals were defined as the background group, and the intermediate individuals were defined as the reference group.

The population of individuals may further include at least one test subject at an unknown stage of the cardiovascular condition, and the one or more individuals for whom the score is calculated comprises the at least one test subject. The data about the target, background and (if present) reference individuals may include information about their state of progression of the condition in order that they can be classified into the target and background groups. However, the method may also be used to determine a stage of the condition for a new test subject that has not been assessed by other means. In this case, the one or more test subjects are included in the population, but would not be part of the target group or background group.

The feature data 10 comprises a plurality of features for each individual, including a plurality of cardiovascular image features. As shown in FIG. 2, the method further comprises a step S210 of determining the cardiovascular image features from images 70 from each of the respective individuals. However, it is not essential that the method comprise this step, as the feature data 10 could be received from the output of a separate method or system that produces the feature data 10.

In the examples discussed below, the plurality of cardiovascular image features are determined from echocardiogram images. Alternatively or additionally, the plurality of cardiovascular image features may be determined from cardiac images, for example images taken using X-ray, computed tomography (CT), magnetic resonance imaging (MRI), positron emission tomography (PET), or any other suitable imaging technique.

As shown in FIG. 2, the feature data 10 further comprises clinical data 80 about each of the respective individuals. This is not essential, and in some embodiments, only the cardiac image features may be included in the feature data 10. The clinical data 80 may comprise one or more of: an age of the individual, a sex of the individual, an ethnicity of the individual, a height of the individual, a weight of the individual, and a medication regime of the individual.

The method further comprises a step S10 of pre-processing the feature data 10 to obtain processed feature data 20. The following steps S20 of applying contrastive principal component analysis and S30 of applying the transformation are performed using the processed feature data 20. FIG. 3 shows further detail of the step S10 of pre-processing the feature data 10. The step S10 of pre-processing the feature data 10 may be omitted in some embodiments, depending on, for example, the quality or origin of the feature data 10.

The step S10 of pre-processing the feature data 10 further comprises S22 imputing missing values for one or more of the features for one or more of the individuals. The imputation of missing values generally precedes the other pre-processing steps when it is present, but this is not essential. The imputation may be performed by interpolating across similar other individuals from the population, or based on others of the features for the same individual. For example, missing values may be replaced with imputed values by using a trimmed scores regression (TSR) tool. Individuals with large numbers of missing values in their corresponding feature data 10 may be excluded from the processed feature data 20, as imputation may be unreliable if too many values are missing. For example, data from individuals with missing values for more than 50%, optionally more than 40%, optionally more than 30%, optionally more than 20% of the features may be excluded from the processed feature data 20.

The step S10 of pre-processing the feature data 10 comprises S24 adjusting the feature data 10 to account for one or more confounding factors. This step is advantageous where different conditions (e.g. technical procedures used during data recording) may affect the feature data 10. Such differences in conditions may thereby affect the quantitative comparison of observations and subsequent identification of relevant biological components. The confounding factors may comprise one or more of a sex of each of the individuals, an age of each of the individuals, a condition under which the feature data was measured, and a medication regime of each of the individuals. For instance, in the examples below, each value in the feature data 10 was adjusted for sex using robust additive linear models with pair-wise interactions [19]. The adjustment for confounding factors may be applied to either or both of the cardiovascular image features and the clinical data 80. An example of the process for adjusting the feature data 10 for confounding factors is shown in panel (i) of FIG. 4.

The step S10 of pre-processing the feature data comprises S26 selecting a subset of the features based on a comparison for each feature of a local variance of the feature with a global variance of the feature. For high-dimensional datasets (e.g. containing considerably more features than observations), it may be desirable to perform an initial selection of features most likely to be involved in a trajectory across the entire population. Any suitable method for preselection may be used, but one method of implementing this selection is the unsupervised method proposed by Welch et al. [20]. This method does not require prior knowledge of features involved in the process. Features are scored by comparing sample variance and neighbourhood variance. A threshold is applied to select those features with higher score. For example, features with at least a 80% probability, optionally a 90% probability, optionally a 95% probability of being involved in a trajectory may be retained. This will correspondingly reduce the dimensionality of the processed feature data 20 compared to the feature data 10. For example, retaining only the features with a 95% probability with mean the processed feature data 20 has a dimensionality around 5% of the dimensionality of the feature data 10.

An example of selecting a subset of the features using the unsupervised method proposed by Welch et al. [20] is shown in panel (ii) of FIG. 4. Features are scored by comparing subject variance and “neighbourhood variance” as:

$${S}_{f}^{2 ( N )} = \frac{1}{{N}_{features} {K}_{c} - 1} \cdot {\sum }_{i = 1}^{{N}_{subjects}} {\sum }_{j = 1}^{{K}_{c}} {( {e}_{if} - {e}_{N ( ij ) f} )}^{2} \qquad ( 1 )$$

where N_{features} is the total number of features, e_{if} is the value of the f^{th} feature in the i^{th} individual, N(i, j) is the j^{th} nearest neighbour of subject i, and K_{c} is the minimum number of neighbours needed to yield a connected graph. S_{f}^{2(N)} is similar to the individual variance computed with respect to neighbouring points rather than the mean, measuring how much f varies across neighbouring individuals.

Intuitively, features most likely to be involved in a trajectory should present a more gradual variation across neighbouring points than at global scale, which would correspond to a high ratio σ_{f}²/S_{f}^{2(N)}. Thus, a threshold is applied to select those features with higher σ_{f}²/S_{f}^{2(N)} score.

The step S10 of pre-processing may comprise any combination of one or more of the steps S22, S24, and S26 depending on the particular implementation and the feature data 10 that is to be used.

The method further comprises a step S20 of applying contrastive principal component analysis between feature data 10 from the background group of individuals and feature data 10 from the target group of individuals to obtain a transformation 30 into a reduced representation space. Where the feature data 10 is pre-processed, the contrastive principal component analysis will be applied between the processed feature data 20 from the background group of individuals and the processed feature data 20 from the target group of individuals.

The contrastive principal component analysis is applied between the feature data 10 from the background group and the target group. Therefore, if the population comprises a reference group, the feature data from the reference group is not included in the contrastive principal component analysis. The method will only use the defined background group and target group to obtain the transformation 30. However, as discussed further below, the transformation 30 will be still applied to all the individuals in the population, including any in the reference group, if present. Thereby, the method detects enriched patterns in the population, while adjusting by confounding components in the background population (i.e. individuals free of the main effect of interest).

The contrastive principal component analysis (cPCA) used herein is similar to that in [21]. cPCA is an example of a dimensionality reduction technique. The high-dimensional feature data 10, in which each feature represents a dimension, is reduced to a lower-dimensional reduced representation space. cPCA returns a number of contrastive principal components (cPCs) that represent the axes of the reduced representation space. By controlling the effects of characteristic patterns in the background (e.g. pathology free and spurious associations, noise), cPCA and its non-linear version contrastive kernel principal component analysis (ckPCA) allow the detection and visualisation of specific data structures that may be missed by other common data exploration and visualisation methods (e.g. non-contrastive PCA or Kernel PCA, t-SNE, UMAP). Before applying the cPCA for contrasted dimensionality reduction, the features in the feature data 10 may be ‘boxcox’ transformed (see

https://www.ime.usp.br/˜abe/lista/pdfQWaCMboK68.pdf), centred to have mean 0, and/or scaled to have standard deviation 1.

cPCA and ckPCA identify low-dimensional patterns that are enriched in the individuals of the target group (i.e. the diseased individuals) relative to the individuals of the background group (i.e. healthy individuals, preferably demographically matched).

The step S20 of applying contrastive principal component analysis comprises applying a contrast parameter to the feature data 10 from the background group. This is not essential, but is preferred in order to improve the differentiation between the target group and background group. Specifically, if C_{target} and C_{background} are the covariance matrices of the feature data 10 from the target group and background group respectively, the cPCs returned by cPCA are the singular vectors of the weighted difference of the covariance matrices: C_{target}−α˜C_{background}, where α is the contrast parameter.

The contrast parameter a represents the trade-off between having high target variance and low background variance. When α=0, cPCA returns cPCs that only maximize the target variance. This effectively reduces to normal, non-contrastive PCA applied on the target data xᵢ (the feature data from the target group). As α increases, directions with smaller background variance become more important and the returned cPCs are driven towards the null space of the background data yᵢ (the feature data from the background group). In the limiting case α=∞, any direction not in the null space (yᵢ) receives an infinite penalty. In this case, cPCA corresponds to first projecting the target data onto the null space of the background data, and then performing PCA on the projected data.

A specific implementation of the cPCA algorithm suitable for the present method is as follows. Other implementations may be used as appropriate for the specific circumstances. For the d-dimensional target data {xᵢ∈ [symbol] ^{d}} and background data {yᵢ∈ [symbol] ^{d}}, let Cₓ, C_{y} be their corresponding empirical covariance matrices. Let [symbol] ᵤₙᵢₜ^{d} [symbol] {v∈ [symbol] ^{d}:∥v∥₂=1} be the set of unit vectors. For any direction v∈ [symbol] ᵤₙᵢₜ^{d}, the variance it accounts for in the target data and in the background data can be written as:

$$Target data variance : {λ}_{x} ( v ) {=}^{def} {v}^{T} {C}_{x} v , \qquad ( 2 )$$

$$Background data variance : {λ}_{x} ( v ) {=}^{def} {v}^{T} {C}_{y} v ,$$

Given a contrast parameter α≥0 that quantifies the trade-off between having high target variance and low background variance, cPCA computes the contrastive direction v* by optimizing

$${v}^{*} = arg {max}_{v ∈ {ℝ}_{unit}^{d}} {λ}_{x} ( v ) - a {λ}_{y} ( v ) . \qquad ( 3 )$$

This problem can be rewritten as

$${v}^{*} = arg {max}_{v ∈ {ℝ}_{unit}^{d}} {v}^{T} ( {C}_{x} - α {C}_{y} ) v , \qquad ( 4 )$$

which implies that v* corresponds to the first eigenvector of the matrix C [symbol] (Cₓ−αC_{y}) Hence the contrastive directions defining the axes of the reduced representation space can be efficiently computed using eigenvalue decomposition. Analogous to PCA, the leading eigenvectors of C are referred to as the contrastive principal components (cPCs). These are the contrastive directions used as the axes of the reduced representation space. The cPCs are eigenvectors of the matrix C and are hence orthogonal to each other. Thereby, for a fixed a, the optimisation (1) is computed, and returns the reduced representation space spanned by the first few cPCs. Typically the first two cPCs are used, but in general any number of cPCs may be used, for example the first three cPCs, optionally the first four cPCs, optionally the first five or more cPCs.

In some embodiments, the step S20 of applying contrastive principal component analysis comprises applying kernel contrastive principal component analysis, such that the transformation 30 into the reduced representation space is non-linear. Normal cPCA returns a linear transformation 30, but kernel cPCA can allows for more complex dependences of the transformation 30. Kernel cPCA can be derived as follows [18].

Consider a nonlinear transformation Φ: [symbol] ^{d}→F that maps data to some reduced representation space F. Firstly, the case where the mapped data is centred is considered. The uncentred case will be considered below. In the centred case, Σᵢ₌₁ⁿΦ(xᵢ)=Σⱼ₌₁ⁿΦ(yᵢ)=0, where Φ(x₁), . . . , Φ(xₙ), and Φ(y₁), . . . , Φ(yₘ) are mappings of the target data xᵢ and background data yᵢ into the reduced representation space respectively. The covariance matrices for the target data and background data are

$$A = \frac{1}{n} {\sum }_{i = 1}^{n} Φ ( {x}_{i} ) Φ {( {x}_{i} )}^{T} , \qquad ( 5 )$$

$$B = \frac{1}{n} {\sum }_{j = 1}^{n} Φ ( {y}_{j} ) Φ {( {y}_{j} )}^{T} .$$

The contrastive components should satisfy

$$λ v = ( A - α B ) , \qquad ( 6 )$$

where the k-th eigenvector corresponds to the k-th contrastive principal component. Let N=n+m, and define the data z₁, . . . , z_{N} as

$${z}_{i} = \{ \begin{matrix}{x}_{i ,} & if 1 \leq i \leq n \\ {y}_{i - n} & otherwise\end{matrix} . \qquad ( 7 )$$

As all contrastive principal components v lie in the span of Φ(z₁, . . . , z_{N}), there exists α=(α₁, . . . , αᵢ)∈ [symbol] ^{N} such that v can be written as

$$v = {\sum }_{k = 1}^{N} {α}_{k} Φ ( {z}_{k} ) . \qquad ( 8 )$$

Also, instead of (6), consider the following equivalent system (9)

$$λΦ ( {z}_{i} ) \cdot v = Φ ( {z}_{i} ) \cdot ( A - α B ) v , i = 1 , … , N . \qquad ( 9 )$$

Substituting (8) into (9), we have

$$λΦ ( {z}_{i} ) \cdot {\sum }_{k = 1}^{N} {α}_{k} Φ ( {z}_{k} ) = Φ ( {z}_{i} ) \cdot ( A - α B ) {\sum }_{k = 1}^{N} {α}_{k} Φ ( {z}_{k} ) , for i = 1 , … , N . \qquad ( 10 )$$

Define the N×N kernel matrix K by

$${K}_{ij} = Φ ( {z}_{i} ) \cdot Φ ( {z}_{j} ) , \qquad ( 11 )$$

and further define the N×N matrices K^{A}, K^{B} by

$${K}_{ij}^{A} = \{ \begin{matrix}{K}_{ij} & if 1 \leq i \leq n \\ 0 & otherwise\end{matrix} , \qquad ( 12 )$$

$${K}_{ij}^{B} = \{ \begin{matrix}0 & if 1 \leq i \leq n \\ {K}_{ij} & otherwise\end{matrix} ,$$

Stacking all N equations together, the left-hand side of (10) is equal to λKa. In addition, the right-hand side is equal to

$$K ( \frac{1}{n} {K}^{A} - \frac{a}{m} {K}^{B} ) a .$$

The linear system (10) can be rewritten as

$$λ Ka = K ( \frac{1}{n} {K}^{A} - \frac{a}{m} {K}^{B} ) a . \qquad ( 13 )$$

The solution of (13) can be found by solving the eigenvalue problem

$$λ a = ( \frac{1}{n} {K}^{A} - \frac{a}{m} {K}^{B} ) a \qquad ( 14 )$$

for non-zero eigenvalues. Clearly all solutions of (14) do satisfy (13). Also, the solutions of (14) and those of (13) differ up to a term lying in the null space of K. Since the projection of the data on v is

$${[ Φ ( {z}_{1} ) \cdot v , … , Φ ( {z}_{N} ) \cdot v ]}^{T} = Ka , \qquad ( 15 )$$

any term lying in the null space of K does not affect the projected result. Hence solving (14) is equivalent to solving (13). To impose the constraint that ∥v∥=1, the following constraint is applied

$${a}^{T} Ka = 1. \qquad ( 16 )$$

Finally, the projection of the data onto the q-th contrastive principal component can be written as Ka^{(q)} as (15).

The above assumes that the mapping of the background data and target data is centred. The centring assumption can be dropped as follows. Assume that Φ(xᵢ) and Φ(yⱼ) have some general mean

$${μ}_{x} = \frac{1}{n} {\sum }_{i = 1}^{n} Φ ( {x}_{i} ) and {μ}_{y} = \frac{1}{n} {\sum }_{j = 1}^{n} Φ ( {y}_{j} ) .$$

Let the non-centred kernel matrix K be the same as (11), and let it be partitioned into

$$K = [ \begin{matrix}{K}_{X} & {K}_{XY} \\ {K}_{YX} & {K}_{Y}\end{matrix} ] , \qquad ( 17 )$$

according to if the elements zᵢ and zⱼ belong to the target or the background data. Then the kernel matrix K can centred as

$${K}_{centre} = [ \begin{matrix}{K}_{X , centre} & {K}_{XY , centre} \\ {K}_{YX , centre} & {K}_{Y , centre}\end{matrix} ] , \qquad ( 18 )$$

$$where$$

$${K}_{X , centre} = {K}_{X} - {1}_{n} {K}_{X} - {K}_{X} {1}_{n} + {1}_{n} {K}_{X} {1}_{n} \qquad ( 19 )$$

$${K}_{XY , centre} = {K}_{YX} - {1}_{m} {K}_{YX} - {K}_{YX} {1}_{n} + {1}_{m} {K}_{YX} {1}_{n}$$

$${K}_{YX , centre} = {K}_{YX} - {1}_{m} {K}_{YX} - {K}_{YX} {1}_{n} + {1}_{m} {K}_{YX} {1}_{n}$$

$${K}_{Y , centre} = {K}_{Y} - {1}_{m} {K}_{Y} - {K}_{Y} {1}_{m} + {1}_{m} {K}_{Y} {1}_{m}$$

and 1ₙ and 1ₘ has all elements-and-respectively.

It can be challenging to get kernel cPCA to work effectively in practice. This is because kernel cPCA is implicitly performing cPCA in the reduced representation space. However, the kernel generally induces a reduced representation space with many correlated features, creating a large null space in the background data. Since cPCA does not have a penalty for directions in this null space and this null space is large, the background dataset will not be very effective at cancelling out directions in the target.

FIG. 5 shows further detail of the step S20 of applying contrastive principal component analysis. The step S20 of applying contrastive principal component analysis comprises applying S31 the contrastive principal component analysis a plurality of times using different values of the contrast parameter to obtain a plurality of different transformations 35, and selecting one of the plurality of transformations 35, wherein the step S30 of applying the transformation uses the selected transformation 30.

As discussed above, the contrast parameter affects the separation of the background data and target data in the reduced representation space. Optimising the contrast parameter can therefore improve the performance of the method and the accuracy of the score. It is not essential that the steps shown in FIG. 5 are used. In some embodiments, a single value of the contrast parameter may be chosen, for example by the user. However, these steps are preferred to optimise the contrast parameter.

Multiple values of the contrast parameter α are used, for example 10 different values, optionally 50 different values, optionally 100 different values, optionally 500 different values. The values may be linearly spaced between an upper and lower bound, or spaced by another method such as logarithmic spacing. In the example, 100 values of a are used logarithmically spaced between 10⁻² and 10². The reduced representation spaces corresponding to each of the plurality of transformations 35 for all the α-values are clustered based on their proximity in terms of the principal angle and spectral clustering [22, 23]. A few of the reduced representation spaces that are far away from each other in terms of the principal angle are selected. The background data and the target data are then projected onto each of these few subspaces, revealing different trends within the target data 10. In some embodiments, selecting one of the plurality of transformations 35 may be performed manually. The appropriate value of α and the corresponding transformation 30 may be manually selected by a user by visually examining the scatterplots that are returned.

However, selecting one of the plurality of transformations 35 preferably comprises automatically selecting one of the plurality of transformations 35, as shown in FIG. 5. Preferably, the transformation is selected that corresponds to the reduced representation space that maximizes the clustering tendency in the projected target data, relative to the clustering tendency in the background data. Automatically selecting one of the plurality of transformations 35 comprises for each of the plurality of transformations 35: determining S33 positions of each of the individuals of the population in a reduced representation space using the transformation; assigning S35 each position to one of a plurality of clusters in the reduced representation space; and calculating S37 a clustering parameter using the positions, the clustering parameter comparing a dispersion within each of the clusters to a reference distribution; selecting S39 a transformation 30 from the plurality of transformations 35 based on the clustering parameter.

Any appropriate method of clustering may be used in the step S35 of assigning each position to one of a plurality of clusters in the reduced representation space. For example, the positions may be clustered using k-means clustering. The optimal number of clusters is determined using a clustering parameter such as the ‘gap’ statistic. The gap statistic compares the change in within-cluster dispersion with that expected under an appropriate reference null distribution [25]. The step S39 of selecting a transformation comprises selecting the transformation that has the optimal number of clusters (or a number of clusters closest to the optimal number) in the reduced representation space based on the clustering parameter, i.e. the gap statistic.

The optimal number of clusters may be determined in any suitable manner. The number of clusters may be chosen as the number of clusters at which an ‘elbow point’ is reached, where adding further clusters no longer results in a significant increase in the variance explained by the clusters. For example, the number of clusters at which adding a further cluster results in a decrease in within-cluster dispersion that is below a predetermined threshold, or at which the rate of change of within-cluster dispersion (i.e. the gap statistic) has a maximum.

In the examples below, when cPCA was applied to the subset of features selected in step S26, approximately six to eight contrasted principal components were obtained capturing the most enriched pathological properties in the target group relative to the background group (where the background group comprised individuals with normal blood pressure measures and not on pharmacological therapy).

The transformation 30 is used in two different ways in the method, according to different aspects. The first aspect corresponds to the left-hand branch of FIG. 1, comprising steps S30-S50. The second aspect corresponds to the right-hand branch in FIG. 1, comprising steps S60 and S70. These two aspects will be discussed in more detail below. In FIG. 1, both aspects are combined and performed in parallel. However, in general, the method may comprise either or both of the two aspects of the flowchart of FIG. 1. If both aspects are present, the two aspects may be performed in any order, and can be performed sequentially or in parallel.

The method comprises a step S30 of applying the transformation 30 to the feature data 10 from the population of individuals to determine a position of each individual of the population in the reduced representation space. This effectively comprises projecting the high-dimensional feature data 10 into the lower-dimensional reduced representation space. Any suitable projection method may be used.

The method then comprises a step S40 of determining trajectories 40 in the reduced representation space between the target group and the background group by connecting the positions of the individuals of the population in the reduced representation space.

The method comprises a step S35 of calculating a matrix of distances among the positions of the individuals of the population in the reduced representation space. The step S40 of determining trajectories is performed on the basis of the matrix of distances. The distances are Euclidean distances, but other distance measures may be used, for example a distance measure weighted by the region of the reduced representation space.

The step S40 of determining trajectories comprises determining a minimum spanning tree among the positions of the population in the reduced representation space based on the matrix of distances. Other structures for connecting the positions of the individuals of the population may be used in other embodiments. For example, other types of spanning trees may be used. Any suitable algorithm may be used to determine the connections among the positions of the individuals. The step S40 comprises defining the trajectories as paths within the minimum spanning tree. The minimum spanning tree is used to calculate the shortest trajectory from any individual to the background group. By connecting the individuals in this way, each specific trajectory consists of the concatenation of relatively similar individuals, with a given behaviour in the reduced representation space. This allows the method to identify similar paths of the progression of the condition using similar individuals.

FIG. 6 shows an example of applying the transformation to the feature data 10 and determining trajectories 40. The example reduced representation space is defined by three cPCs labelled cPC1, cPC2, and cPC3. Several example individuals 45 are shown who are at the end of a trajectory 40. As can be seen in FIG. 6, each trajectory comprises a series of straight-line segments in the reduced representation space. This is because the trajectories 40 are determined by connecting the positions of the individuals, i.e. so that each vertex of each trajectory is a position of an individual. The illustrated example individuals 45 are merely the individuals at the ends of the trajectories 40. FIG. 6 is merely illustrative, and in implementations of the method, it is likely that there would be a much larger number of trajectories 40 than illustrated in FIG. 6.

The cPCA allows each individual to be represented in the reduced representation space associated with the condition, where the corresponding position reflects the individual's pathological state. In FIG. 6, proximity to the bottom-left corner (where the background group is located) implies a pathology-free state. Conversely, the top-right corner (where the target group would be located) implies a more advanced progression of pathology. For visualization simplicity, FIG. 6 only shows a space represented by the first three cPCs, but the quantitative analysis considers all identified cPCs where there are more than three.

Within this reduced representation space defined by the cPCs, each individual is automatically assigned to a condition trajectory. The trajectories 40 represent corresponding subpopulations of subjects potentially following a common condition variant, i.e. following a particular path through the reduced representation space from the pathology-free state to a more advanced pathological state. The number of subpopulations (condition trajectories) is determined automatically based on how the individuals “cluster” together in the reduced representation space, i.e. how the positions of the individuals are connected together to form the trajectories.

The trajectories 40 can be used for subtyping of individuals according to the proximity to the background group in the reduced representation space. The step S40 of determining trajectories 40 further comprises identifying one or more subtrajectories representing paths in the reduced representation space based on the matrix of distances. Each subtrajectory comprises a plurality of the trajectories 40, as shown in FIG. 6. The step S40 comprises assigning each individual of the population to one or more of the subtrajectories. As discussed, each trajectory 40 represents a particular path through the reduced representation space. Similar trajectories may be grouped together and used to identify sub-types of the condition.

The identifying of the one or more subtrajectories may comprise performing spectral clustering over the matrix of distances. Spectral clustering [22] is performed over the cPC-based matrix of Euclidean distances to identify individuals' subtrajectories in the reduced representation space. Some individuals may be assigned to multiple subtrajectories, thereby implying that the subtrajectories may overlap. Assignment to multiple subtrajectories is particularly possible in the early stages of the condition, either due to the algorithm being unable to distinguish between different paths, or due to real biological effects (e.g., two disease variants with a common or similar starting process).

Once the trajectories 40 have been determined, these can be used to determine the score 50. The method comprises, for one or more of the individuals of the population, calculating the score 50 as a distance along the one of the trajectories 40 on which the position of the individual lies. Since the trajectories 40 represent paths from the pathology-free state to more advanced pathological states, an individual's position along the trajectory is a measure of the progression of the condition for that individual.

As shown in FIG. 6, the trajectories 40 connect to a reference point 41. For example, each trajectory may have one of its endpoints at the reference point 41. The distance along the one of the trajectories 40 on which the position of the individual lies is a distance between the position of the individual and the reference point 41 along the trajectory. Using a common reference point 41 to define one end of the trajectories improves the comparability of the scores between individuals whose positions lie on different trajectories 40.

The reference point 41 is an average position in the reduced representation space of individuals in the background group, as shown in FIG. 6. This means that a larger distance and correspondingly larger score 50 represent a more advanced progression of the condition. The position of each individual in their corresponding trajectory 40 reflects the individual proximity to the pathology-free state (indicated by the background group) and, if analysed in the inverse direction, to advanced condition progression. Thus, to quantify the distance to these two extremes (background or having the condition), a score 50 is calculated as the shortest distance value to the background's centroid or average position.

In some embodiments, other positions in the reduced representation space may be used as the reference point 41. For example, an average position of individuals in the target group may be used. In such an embodiment, a larger distance and corresponding larger score 50 would indicate greater distance from the pathological state, and therefore a less advanced condition progression. To make the score 50 easier to interpret, it may be normalised. For example, the score 50 may be normalised relative to the maximum value for the population, i.e. so that the normalised values are standardised between 0 and 1.

The method comprises a step S60 of calculating a contribution of each of the plurality of features to the transformation 30. This allows the evaluation of which features are most informative about the cardiovascular condition.

Calculating the contribution comprises, for one or more principal components from the contrastive principal component analysis, calculating a product of an eigenvalue of the principal component with a loading of the feature for the principal component. The contribution for the feature comprises a sum of the products. The one or more principal components comprises principal components having an eigenvalue above a predetermined value. This enables the method to exclude rapidly features that have a small contribution, which simplifies the subsequent analysis. For example, the predetermined value may be 0.01, optionally 0.025, optionally 0.05. The product is normalised by a sum for the principal component of the loadings of the plurality of features.

Specifically, the total contribution Cᵢ of each feature i to the obtained reduced representation space (and the corresponding trajectories 40) is quantified as [26]

$${C}_{i} = 100 {\sum }_{j = 1}^{{N}_{cPC}} ( {λ}_{j}^{norm} \frac{{ω}_{i . j}^{2}}{{\sum }_{k = 1}^{{N}_{features}} {ω}_{k . j}^{2}} ) , \qquad ( 20 )$$

where λⱼ^{norm}=(λⱼ=min λ)/Σₖ₌₁^{N}^{ₜₒₜₐₗ}(λₖ−min λ) is the normalized eigenvalue of the contrasted principal component j, min λ is the minimum obtained eigenvalue, Nₜₒₜₐₗ is the original number of contrasted principal components, N_{cPC} is the number of contrasted principal components with λⱼ^{norm} over a predefined value (in this case 0.025), ω_{i,j} is the loading/weight of the feature i on the component j, and N_{features} is the total number of features considered in the contrastive principal component analysis.

The method comprises a step S70 determining a plurality of the features having the highest contributions to the transformation 30. For example, the method may select the 5 features, optionally 10 features, optionally 15 features, optionally 25 features having the highest contribution. Alternatively, the method may select all features having a contribution above a second predetermined value, which may be different from the predetermined value used for comparison to the eigenvalues of the principal components discussed above.

FIG. 7 shows a method of determining a subject score 55 representative of a progression of a cardiovascular condition for a test subject. As mentioned above, a score for a new individual may be obtained by including the test subject in the population used for the method of FIG. 1. The test subject is then included in the determination of the trajectories 40, and the score for the test subject determined. Alternatively, the method of FIG. 7 may be used to determine the subject score 55 for the test subject.

The method comprises a step S110 of pre-processing the subject feature data 15 from the test subject to obtain processed subject feature data 25. As for the target data and background data, the subject feature data 15 comprises data on the plurality of features for the test subject, including a plurality of cardiovascular image features. The step S110 of pre-processing is substantially the same as described above for the methods of FIG. 1. As for FIG. 1, the step S110 of pre-processing may be omitted in some embodiments.

The method comprises a step S120 of determining a position of the test subject in a reduced representation space by applying a transformation 30 into the reduced representation space to the subject feature data from the test subject. The transformation 30 may be obtained using an embodiment of the method described above. The position of the test subject may be determined by projecting the subject feature data 15 into the reduced representation space as for the feature data from the population described above.

The method comprises determining a position of the test subject on one of a plurality of trajectories 40 in the reduced representation space. The trajectories 40 may be determined using an embodiment of the method described above. The position of the test subject on the one of the trajectories may be determined as a closest position on one of the trajectories 40, i.e. the nearest point in the reduced representation space to the position of the test subject that lies on one of the trajectories 40. Alternatively, one of the trajectories 40 that is nearest to the position of the test subject may be redefined to include the position of the test subject.

The method comprises calculating the subject score 55 using a position along the one of the trajectories on which the position of the subject lies.

The plurality of features may be a plurality of features having the highest contributions to the transformation determined using an embodiment of the method described above. This may simplify the calculations when calculating subject scores for new test subjects, and also requires a smaller number of features to be measured for the test subject when determining their subject score. Since the plurality of features having the highest contributions determine the bulk of the contribution to the transformation, omitting other features is unlikely to significantly reduce the accuracy of the subject score.

Any of the methods described above may be embodied in a computer program comprising instructions which, when the program is executed by a computer, cause the computer to carry out the method.

As shown in FIG. 1, the method may be carried out by a system 100 for calculating a score representative of a progression of a cardiovascular condition and/or for analysing feature data about a cardiovascular condition. The system 100 comprises a processor configured to carry out the steps of the method. The steps of the method shown in FIG. 1 therefore also represent functional units of the system, which may be, for example, programming functions or dedicated integrated circuits.

Similarly, as shown in FIG. 7, the method of determining a subject score 55 for the test subject may be carried out by a system 200 for determining a subject score representative of a progression of a cardiovascular condition for a test subject. The system 200 comprises a processor configured to carry out the steps of the method. The steps of the method shown in FIG. 7 therefore also represent functional units of the system, which may be, for example, programming functions or dedicated integrated circuits.

The efficacy of the method is demonstrated by the following results.

### Results

The present results use cross-sectional datasets of young adults with a range of blood pressure measures to study the disease progression of hypertension. In this example, the cardiovascular condition is hypertension, so the score determined by the method is referred to as a disease score, or a disease progression score. The method integrates the effect of relevant resting clinical and echocardiography image features to place individuals on a trajectory from health to disease, and thereby determine disease scores for the individuals. In addition, important clinical and echocardiography image features relevant to the disease progression of hypertension in young adults were identified. The changes of individual features over the course of the disease progression were also assessed. The disease score was assessed by evaluating its association with the modified cardiovascular risk score and clinical management stages.

### Study Population

Data was taken from three datasets from the Oxford Cardiovascular Clinical Research Facility in the UK. The studies are Young Adult Cardiovascular Health sTudy (YACHT), Trial of Exercise to Prevent Hypertension in young Adults (TEPHRA), and Hypertension management in Young adults Personalised by Echocardiography and clinical Outcomes (HyperEcho). Only participants recruited before March 2020 were included, and those with known gestational history of preterm birth were excluded from this study. The three datasets were combined after independent data processing and cleaning.

The YACHT study (NCT02103231) was an observational case-control study, started in August 2014 and completed in May 2016 [27]. The aim of this study was to investigate cardiovascular structure and function, and physical exercise response in full-term born (≥37 weeks), prematurely born (<37 weeks), and hypertensive young adults aged 18 to 40 years. The study was approved by the South Central Berkshire Research Ethics Committee (Reference 14/SC/0275).

The TEPHRA study (NCT02723552) was a single centre, two-arm, and parallel randomised controlled (1:1) trial, started in June 2016 and completed in January 2020 [28]. The aim of this trial was to assess the effect of physical exercise on lowering blood pressure measures in young adults (aged 18 to 35 years) with elevated blood pressure. Participants underwent a baseline study visit for detailed assessment of cardiovascular structure and function. Then they were randomised to either a 16-week exercise intervention arm or control arm. Participants randomised to the exercise intervention were provided with a gym membership to complete three supervised aerobic exercise sessions (60 minutes each) per week and for 16 weeks. The control arm participants were advised to maintain their usual physical activity levels. After 16 weeks of randomisation, all participants attended their second assessment visit for a follow-up cardiovascular assessment [28]. TEPHRA was approved by the Oxford B Research Ethics Committee (Reference 16/SC/0016).

The HyperEcho study (NCT03762499) is a multi-centre longitudinal observational study, started in October 2018 and still ongoing with an expected completion to be in 2028. The aim of this study is to improve and personalise the management of young adults with hypertension. Participants are characterised as hypertensive patients aged between 18 to 40 years old and referred to an NHS hypertension clinic in England to manage their blood pressure. The study has been conducted to investigate whether baseline transthoracic echocardiography imaging along with routine clinical data collected in the hypertension clinic can improve risk stratification for cardiovascular disease in young adults with hypertension. The study was approved by the South West-Frenchay Research Ethics Committee (Reference 18/SW/0188).

A comprehensive transthoracic two-dimensional (2D) echocardiography scan was performed for each individual using a Philips EPIC 7C, or Philips iE33 echocardiography ultrasound machine (Philips Healthcare, Surrey, United Kingdom) and following the British Society of Echocardiography standards in image acquisition and optimisation [29]. Conventional image analysis was completed offline according to the latest published guidelines for chamber [30] and valvular [31] assessment using Philips IntelliSpace Cardiovascular (ISCV) 2·1 (Philips Healthcare Informatics, Belfast, Ireland), and TomTec Image Arena 4·6 (Chicago, IL, United States) software was used to perform 2D left ventricular and left atrial speckle tracking analysis according to the European Association of Cardiovascular Imaging (EACVI) recommendations [32].

Demographics data including age, sex, height, weight, and body mass index (BMI) were collected from all individuals at their baseline visit. Resting blood pressure measurements were obtained using a digital blood pressure monitor (GE Dinamap V100, GE Healthcare, Chalfont St. Giles, United Kingdom) to record three consecutive blood pressure readings on the left arm with a minute apart. The last two measurements were averaged and included in the analysis. Fasting blood samples (a minimum of four hours fasting) were collected for each participant and sample analysis was carried out at the Oxford John Radcliff Hospital Biochemistry Laboratory.

Anti-hypertension treatment information was collected from the Electronic Patient Record (EPR) system, as well as from the clinical notes with extracting the date of treatment initiation. Participants who were not referred to a clinic, had completed a questionnaire about their medical history and hypertension treatment information.

Between August 2014 and March 2020, 542 young were enrolled to the YACHT, TEPHRA, and HyperEcho studies, of which 131 participants were excluded (n=117 participants with history of premature birth, and n=14 participants with >30% missing data). This resulted in a population of 411 individuals (28·9±5·7 years) with a range of blood pressure measures (94 mmHg, and 68.67 mmHg; the range for systolic and diastolic blood pressure measures, respectively). Half of the cohort are males (51.6%) and the average BMI was 26·29±5. Table 1 illustrates the baseline clinical characteristics of the 411 cohort participants.

TABLE 1
Baseline clinical characteristics for the population of
individuals making up the study cohort. Numeric data is
presented as mean ± standard deviation and categorical
data is presented as number of participants and percentage.

| | |
|---|---|
| Age | 28.93 ± 5.74 |
| Male, n (%) | 209 (51.6) |
| Height (cm) | 173 ± 10.03 |
| Weight (kg) | 79.19 ± 18.5 |
| Body mass index | 26.26 ± 5.01 |
| Body surface area | 1.94 ± 0.21 |
| Systolic blood pressure (mmHg) | 132.24 ± 16.59 |
| Diastolic blood pressure (mmHg) | 81.66 ± 12.82 |
| Cholesterol level (mmol/L) | 4.5 ± 1.11 |
| HDL level (mmol/L) | 1.34 ± 0.32 |
| LDL level (mmol/L) | 2.71 ± 0.79 |
| Triglycerides level (mmol/L) | 1.22 ± 0.87 |
| Cholesterol to HDL ratio | 3.52 ± 1.23 |
| Smokers, n (%) | 45 (11.59) |
| On antihypertension medication, n (%) | 124 (31.47) |

Individuals from YACHT and TEPHRA had a cardiovascular risk score calculated based on eight risk factors, including: body mass index, cardiovascular fitness level, Alcohol consumption, smoking status, blood pressure on awake ambulatory monitoring, blood pressure response to exercise, total cholesterol level, and fasting glucose level. Details of the score calculation and methods for each factor were published in 2018 [27, 28]. Participants were classified into four categories based on their calculated cardiovascular risk score, with lower scores indicate higher risk of cardiovascular disease.

### Method Implementation

The population of individuals comprised the 411 young adults (28·9±5·7 years) with a range of blood pressure measures from the above three studies conducted at the Oxford Cardiovascular Clinical Research Facility in the UK. All participants completed baseline clinical assessment including echocardiography imaging, as above.

The method described above was applied to identify low-dimensional patterns in target individuals with high systolic blood pressure measures (≥160 mmHg) relative to a normotensive background group with lower measures (<120 mmHg). Based on the variance similarities, the individuals were ordered and assigned with a disease score normalised from zero (health) to one (disease). The pattern of remodelling of features having high contributions to the transformation was tested. The effect of anti-hypertension treatment and exercise intervention on the disease score was also investigated.

The method was implemented using MATLAB R2019b programming environment (Mathworks Inc., Natick, MA, USA). After labelling individuals with hypertensives (target group), normotensive (background group), and intermediate (reference group), contrastive principal component analysis (cPCA) was applied to the feature data comprising clinical and echocardiography image features [17]. The method identifies low-dimensional unique patterns in the hypertensive (target) group relative to the normotensive (background) group. The distance between individual participants was measured based on the variance similarities.

Each individual was assigned with a unique location in the reduced representation space and ordered relative to the proximity of the normotensive group. The disease score was calculated as the shortest distance value to the normotensive centroid, and values were standardised between zero and one. Participants with low scores are closer to the normotensive group and those with higher scores are closer to the hypertensive group. TEPHRA participants who were randomised to the exercise intervention arm had another disease progression score generated from data collected during their follow-up visit.

Feature contribution to the transformation was identified based on the extent to which the values differ between subjects of the normotensive and hypertensive groups, relative to the variation within the groups. An unsupervised learning feature-selection method was applied to identify highly contributed features based on a certain threshold value. The threshold is called the expected contribution, which was measured by comparing variances between individuals.

### Disease Score Validation

To test the disease score robustness and stability, two criteria for stability and validity were applied.

Stability is achieved when, if a few participants are excluded from the model, the disease scores for the remaining participants do not change significantly. To test stability, after applying the method on the full population dataset and obtaining disease scores (original) for each participant, a 5K cross-validation test was carried out to divide the dataset into 80% for training and 20% for testing. This was shuffled and repeated five times. The Root Mean Squared Deviation (RMSD) was then calculated by measuring the differences between repeated and original values. The differences were squared, and the sum of the squared differences was divided by the number of subjects and then the square root was calculated. An RMSD value of ≥0·5 was considered as an indication of poor model stability.

Validity is the ability to differentiate between pathology-free participants and those with more advanced pathology. The differences in disease scores between the hypertensive and normotensive groups were tested using independent-samples t-test. A p-value of ≤0·05 was used to indicate statistical significance and acceptable performance. The method should be valid when the normotensive participants have lower disease scores compared with the hypertensive participants. Failing to meet the above criteria would indicate that the disease scores are not valid.

### Post-Hoc Statistical Analyses

R 4.0.2 and R studio programming language was used for post-hoc statistics and graphics. The log 10 method was applied to transform data to approximately a normal distribution. To assess the pattern of changes through the disease progression for individual features, the disease progression scores were divided into ten consecutive subgroups. Participants with score 0-0·25 were in the first group, and then each group consisted of 20 consecutive participants. The first three groups were categorised as a low score (disease progression score from 0 to <0·3), medium score was for groups from four to seven (disease progression score from ≥0·3 to <0·5), and high score represents groups from eight to ten (disease progression score ≥0·5). Variables were scaled between zero and one to allow relative comparison.

Participants were classified based on their clinical stage of hypertension in four categories: no referral or treatment, referred with no treatment, referred with less than two years treatment, and referred with more than two years treatment. One-way ANOVA test was applied to determine the disease progression score difference between the four categories, and the cardiovascular risk score groups. Pearson correlation test was used to test the relationship between the change in disease progression score and fitness variables. A p-value of ≤0·05 was used to indicate statistical significance and a 95% confidence interval was used.

The plurality of features in the feature data used included 68 clinical and echocardiography variables (age, BMI, and 66 echocardiography variables), which are listed in Table 2. Variables with more than 30% missing data were excluded (n=7). After the contrastive dimensionality reduction of the data (using contrastive principal component analysis), variables with the highest weight have the highest contribution for the transformation. Each participant was assigned to a location on the reduced representation space with a disease progression score according to the shortest path along their trajectory to the normotensive centroid. The relationship between the disease progression scores and clinical systolic blood pressure for all participants is shown in FIG. 8.

FIG. 8 is a scatter plot demonstrating the relationship between disease scores and clinical systolic blood pressure for all individuals. The green dots represent individuals in the background group (healthy). Red dots represent individuals in the target group (hypertensives). The grey dots represent individuals in the reference group. The reference group was not involved in the contrastive principal component analysis. However, the reference individuals were given disease scores based on their similarities and the distance to the background group.

TABLE 2
Variables included for the disease progression model development.

1. Age (years)
2. Body mass index
3. Heart rate (bpm)
4. Interventricular septum (cm)
5. LV internal diastolic dimension (cm)
6. LV posterior wall thickness (cm)
7. LV internal systolic dimension (cm)
8. LV ejection fraction, Teichholz (%)
9. LV outflow tract (cm)
10. LV relative wall thickness
11. LV mass (g)
12. LV mass index (g/m²)
13. LV 4-ch end diastolic volume (ml)
14. LV 4-ch end systolic volume (ml)
15. LV 4-ch ejection fraction (%)
16. LV 4-ch stroke volume (ml)
17. LV 2-ch end diastolic volume (ml)
18. LV 2-ch end systolic volume (ml)
19. LV 2-ch ejection fraction (%)
20. LV 2-ch stroke volume (ml)
21. LV biplane end diastolic volume (ml)
22. LV biplane end systolic volume (ml)
23. LV biplane ejection fraction (%)
24. LV biplane stroke volume (ml)
25. LV biplane cardiac output (ml/min)
26. LA 4-ch volume (ml)
27. LA 2-ch volume (ml)
28. LA biplane volume (ml)
29. Mitral valve E velocity (cm/s)
30. Mitral valve A velocity (cm/s)
31. E/A
32. Deceleration time (ms)
33. Lateral S′ velocity (cm/s)
34. Lateral E′ velocity (cm/s)
35. Lateral A′ velocity (cm/s)
36. Septal S′ velocity (cm/s)
37. Septal E′ velocity (cm/s)
38. Septal A′ velocity (cm/s)
39. E′_{average} (cm/s)
40. E/E′ₗₐₜₑᵣₐₗ
41. E/E′ₛₑₚₜₐₗ
42. E/E′_{average}
43. Aortic valve max velocity (cm/s)
44. LVOT velocity time integral (cm)
45. Pulmonary valve max velocity (cm/s)
46. Pulmonary artery acceleration time (ms)
47. RV basal dimension (cm)
48. RV mid dimension (cm)
49. RV length (cm)
50. RA volume (ml)
51. Tricuspid regurgitation max velocity (cm/s)
52. TAPSE (cm)
53. RV S′ velocity (cm/s)
54. RV E′ velocity (cm/s)
55. RV A′ velocity (cm/s)
56. Interventricular contraction time (cm)
57. Interventricular relaxation time (cm)
58. Ejection time (cm)
59. LV Global longitudinal strain (%)
60. LA Peak longitudinal strain, 4-ch reservoir (%)
61. LA Peak contraction strain, 4-ch booster pump (%)
62. LA 4-ch conduit (%)
63. LA Peak longitudinal strain, 2-ch reservoir (%)
64. LA Peak contraction strain, 2-ch booster pump (%)
65. LA 2-ch conduit (%)
66. LA Peak longitudinal strain - biplane reservoir (%)
67. LA Peak contraction strain - biplane booster pump (%)
68. LA biplane conduit (%)
LV, left ventricle; LA, left atrium; 4-ch, four-chamber; 2-ch, two-chamber; LVOT, left ventricular outflow tract; RV, right ventricle; RA, right atrium; TAPSE, tricuspid annular plane systolic excursion.

A total of 21 variables were identified as having the highest contributions to the transformation. This shows the important phenotypes for this cohort. These features contributed more than 80% in total, with the expected contribution calculated at 1·47%. These variables can be grouped in three categories; 1. Left atrial structure and function, 2.Left ventricular volumes, and 3. E Doppler velocities. FIG. 9 illustrates the contribution percentage of the 3 categories with the sum percentage of the remaining variables (47 variables).

FIG. 9 shows the different categories of features having the highest contributions by the percentage of contribution to the total. In this example, the cardiovascular image features were determined from echocardiogram images. Almost half of the contribution to the transformation 30 was from the left atrial (LA) function (41%). The next most significant features were the left ventricular (LV) volumes contributing 23%, and the E velocities contributing 19%. The remaining features contributed 17% of the total. The contributions are also shown in Table 3 below.

TABLE 3
LA, left atrium; LV, left ventricle; EDV, end diastolic
volume; ESV, end systolic volume; SV, stroke volume; bp,
biplane; 4ch, four-chamber view; 2ch, two-chamber view.

| LA 41% | LV 23% | E velocities 19% | Others 17% |
|---|---|---|---|
| Conduit bp - 6.6% | Systolic diameter - 9.2% | E/E′_{Average} - 6.3% | The remaining |
| Conduit 4ch - 6.03% | EDV 2ch - 3.5% | E/E′_{medial} - 4.5% | 47 variables |
| Reservoir bp - 5.5% | EDV bp - 3.4% | E/E′ₗₐₜₑᵣₐₗ - 3.8% | |
| Conduit 2ch - 4.8% | SV 2ch - 2.9% | E′_{medial} - 2.5% | |
| Reservoir 2ch - 4.1% | ESV bp - 2.5% | E′ₗₐₜₑᵣₐₗ - 1.6% | |
| Pump bp - 4.04% | ESV 4ch - 1.7% | | |
| Pump 2ch - 3.1% | | | |
| Reservoir 4ch - 3.1% | | | |
| Volume bp - 2.2% | | | |
| Pump 4ch - 1.6% | | | |

The change in individual variables through the course of the disease progression was studied for contributed variables. FIG. 10 illustrates the pattern of remodelling in individual features (contributed variables) throughout the disease progression, as represented by the disease scores. FIG. 10A is a heat map demonstrating the mean value of each contributed variable throughout the disease progression. The disease progression scores were divided into ten consecutive subgroups. The first three groups were categorised as a low score, medium score was for groups from four to seven, and high score represents groups from eight to ten. Left atrial reservoir and conduit function were the highest in participants with low disease progression score, while the pump function and E/E′ ratio were the highest in those with high score. Left atrial reservoir and conduit function appear to have the same pattern as the E′ medial and lateral velocities, in which they decrease as the disease progresses. In contrast, E/E′ ratios and the left atrial pump function have similar pattern of remodelling. These findings are consistent with previous studies that reported the prognostic value of left atrial function in hypertension as individual parameters for the prediction of adverse cardiovascular events [33, 34].

The radar chart in FIG. 10B illustrates the pattern of remodelling for participants with low, medium, and high disease progression scores based on a selected set of eight echocardiography variables (biplane and average measures). Participants with a low disease progression score (yellow chart) had the highest left ventricular systolic diameter and left atrial reservoir and conduit function and the lowest left atrial pump function, left atrial volume, and E/E′ ratio. In contrast, participants with a high disease progression score (blue chart) had the highest left atrial pump function, and E/E′ ratio and the lowest left atrial conduit, left ventricular diameter and volumes.

The continuous relationship between the disease progression score and left atrial structure and function, left ventricular measures, and E Doppler velocities are illustrated in FIGS. 10C, 10D, and 10E respectively. Left atrial conduit and reservoir function reduces as the disease progression score increases, but with a steeper reduction in the conduit function (FIG. 10C). Left atrial volume appears to increase rapidly until the disease progression score is at 0·4 and then it increases in a slower rate with a maximum increase at score 1. FIG. 10D demonstrates the changes in left ventricular systolic dimeter and left ventricular volumes. All measures have the same pattern of changes through the disease progression score with their peak is at 0·4 but the systolic diameter peaks earlier at 0·25. The change in E Doppler velocities is shown in FIG. 10E with a steep increase of E/E′ ratio after 0·5 and the same pattern of reduction for lateral and medial E′ velocities.

All values were rescaled from zero to one to allow between variables comparison. The abbreviations in FIG. 10 are LV, left ventricle; SV, stroke volume; IDs, internal diameter at end systole; EDV, end diastolic volume; ESV, end systolic volume; LA, left atrium; bp, biplane; 4ch, four-chamber view; 2ch, two-chamber view; med, medial wall; lat, lateral wall; avg, average.

FIG. 11 demonstrates the disease progression score difference among the four stages of clinical hypertension. The baseline clinical characteristics for each group are presented in Table 4. In this cohort of young adults, participants were recruited from different clinical stages of hypertension based on referral to a hypertension clinic and treatment duration. Participants with no referral or anti-hypertension treatment had the lowest disease progression score compared with those who referred to the clinic (p<0·0001). Participants who have been on longer duration of treatment, had higher score (p<0·001) compared with un-treated participants. In addition, participants with longer duration of treatment (two or more years) had higher disease progression score compared to participants with less than two years of treatment (p=0.037). Dashed line represents the difference between the groups and the solid lines for two groups comparison. A group of 15 participants, who were referred to the hypertension clinic, had no treatment information. Therefore, they were excluded from this analysis. The symbols used in FIG. 11 are as follows: **=P value <0.001, *=P value <0.05, NS=P value >0.05.

TABLE 4
Baseline characteristics of participants at four clinical stages of hypertension.

| | Group 0 | Group 1 | Group 2 | Group 3 | |
|---|---|---|---|---|---|
| | n = 246 | n = 24 | n = 70 | n = 56 | P value |
| Age | 26.49 ± 4.28 | 31.71 ± 5.12 | 31.90 ± 6.29 | 34.49 ± 4.87 | <0.001 |
| Male % | 50.8 | 37.5 | 32 | 42.9 | 0.49 |
| SBP | 124.41 ± 11.44 | 143.07 ± 17.67 | 142.63 ± 16.70 | 145.46 ± 16-18 | <0.001 |
| DBP | 75.78 ± 9.56 | 91.90 ± 11.66 | 89.98 ± 13.38 | 89.96 ± 10.94 | <0.001 |
| Height | 173.07 ± 9.18 | 174.95 ± 8.35 | 172.26 ± 11.40 | 173.47 ± 12.31 | 0.74 |
| Weight | 74.04 ± 13.83 | 82.29 ± 14.36 | 86.75 ± 19.84 | 92.95 ± 24.89 | <0.001 |
| BSA | 1.88 ± 0.21 | 2.11 ± 0.14 | 2.05 ± 0.19 | 2.08 ± 0.16 | <0.001 |
| BMI | 24.7 ± 3.8 | 27.13 ± 3.82 | 28.64 ± 5.08 | 30.78 ± 6.07 | <0.001 |

Numeric data is presented as mean±standard deviation and categorical data is presented as number of participants and percentage. Group 0, No referral or treatment; Group 1, Referred with no treatment; Group 2, Referred with treatment <two years; Group 3, Referred with treatment >two years; SBP, systolic blood pressure; DPB, diastolic blood pressure; BSA, body surface area; BMI, body mass index.

To test the effect of the 16-week exercise intervention on the disease progression score, a subgroup from TEPHRA (n=60) who were randomised to the exercise intervention arm had another disease progression score generated from their follow-up data. The change in the disease progression score from baseline to post intervention was associated with changes in ventilatory threshold. The reduction in the disease progression score post exercise intervention was associated with higher changes in the ventilatory threshold levels (p=0·01). Participants with dropped disease progression score after the intervention had higher change in the ventilatory threshold levels (p=0·01) as demonstrated in FIG. 12A. FIG. 12A shows that the reduction of the disease progression score after the 16-week exercise intervention was associated with improved ventilatory threshold from baseline level. FIG. 12B shows that the change in the disease progression score was also correlated with the number of active days spent at the gym (p=0·015). Specifically, a reduction in the score was associated with a higher number of active days at the gym. In FIG. 12, DP is an abbreviation for disease progression score.

To further understand the value of the disease progression score, for a subgroup of the participants (n=179), the score was tested against a modifiable cardiovascular risk score calculated from eight risk factors [27]. The results are shown in FIG. 13, which illustrates the relationship between the disease progression score and the modifiable cardiovascular risk score. Participants with the lowest cardiovascular risk scores had the highest disease progression scores (p<0·0001).

The results of the 5K cross-validation for the model stability showed that when 20% of the dataset was taken off the model for testing, the disease progression scores were not significantly changed. The RMSD was calculated at 0.43. Also, the mean of disease progression scores was different (p<0·0001) between the normotensive and the hypertensive groups, as shown in Table 5. Thus, the validation criteria for the disease progression model performance were met.

TABLE 5
Disease progression scores difference between
the normotensive and hypertensive groups.

| | Normotensive | Hypertensive | |
|---|---|---|---|
| | n = 111 | n = 33 | P value |
| Disease progression score, | 0.103 ± 0.05 | 0.449 ± 0.22 | <0.0001 |
| Mean ± SD | | | |

### Conclusions

The disease progression of hypertension in young adults was studied from single snapshots of echocardiography features without the need of follow up data. The present method extracts pseudo temporal information from high-dimensional cross-sectional datasets. This helps to overcome limitations created by the lack of longitudinal studies. Echocardiography features can be combined to generate a disease progression score that reflects the severity of hypertension in young adults. The score could be used as an alternative non-invasive tool for risk assessment and as a follow-up tool to optimise hypertension management. This method could help clinicians to personalise management of hypertension, particularly in younger patients.

The method identifies enriched patterns of cardiac phenotypes in participants with hypertension relative to normotensives. The effect of relevant multiple clinical and echocardiography features is combined to generate a disease progression score to order participants based on the severity of hypertension.

A similar computational method was applied to neurodegenerative conditions to predict the stage of neuropathological severity in the spectrum of late-onset Alzheimer's and Huntington diseases from gene expressions [17] and to cancer research to study the dynamic biological and pathological mechanisms [15, 16]. In the present method, the method has been applied on clinical cardiovascular echocardiography-based features for the first time. Due to the non-linear nature of cardiac remodelling in hypertension, it has been challenging to study the disease progression without longitudinal follow-up data [35]. The present contrasted trajectory method uses non-linear modelling to generate the disease progression scores and has achieved better performance compared to other dimensionality reduction approaches, such as traditional PCA and novel non-linear Uniform Manifold Approximation and Projection [17].

A number of recent studies have demonstrated that a combination of parameters can hold more value than single parameters by showing an additional prognostic value of the combined effects of multiple echocardiography features in patients with hypertension using machine learning tools [36-38] and improvement of diagnosis and understanding of disease in patients with heart failure [39]. Therefore, the strength of this method also lies in the combination of echocardiography features, including 2D images, Doppler velocities, and speckle tracking features in this hypertension progression score.

Management of hypertension in patients below the age 40 has been highly conservative due to the insufficient longitudinal data in the literature about the effect of anti-hypertension treatment for this group of patients [6]. Machine learning tools have been also applied to combine 47 continuous echocardiography, clinical, and laboratory variables, to cluster hypertensive patients into distinct groups that may benefit from targeted treatment plans [36].

This method overcomes the dataset limitation by developing the disease progression model from cross-sectional features. Furthermore, when exercise intervention was considered, the reduction in the score post exercise intervention was associated with improved fitness levels. Such a score could help clinicians to improve and personalise the management plan for hypertension in younger patients. Thus, as echocardiography is a non-invasive and widely available tool, using the disease progression score may lead to reduce the number of investigations requested for young hypertensives. The disease progression score was in line with the cardiovascular risk score, this method could provide an alternative approach for risk assessment using echocardiography imaging, without the need of blood samples or exercise testing.

In the example above, a relatively small study sample was used to develop the disease progression score. Performance could be improved with a larger cohort of young adults. The majority of participants (>97%) included in the population above were recruited at a single centre. Preferably, when implementing the method, a wider range of individuals should be included to reduce the chance of bias in the disease score.

Some of the features having a high contribution to the transformation are not often obtained in clinical practice, such as left atrial strain assessment. This could be a limitation in implementation, but it is expected that adequate results could be obtained without including this feature.

In the above, a high cut-off value of systolic blood pressure (≥160 mmHg) was used to define the target group in order to clearly differentiate hypertensive participants from normotensives. However, some individuals in the intermediate group were diagnosed with hypertension and on anti-hypertension treatment. This indicates that lower cut-off values for the target group could be still be used and return valid results.

### Alternative Method

FIG. 14 is a flowchart of an alternative method of calculating a subject score 55 representative of a progression of a cardiovascular condition for a test subject. The steps of the method shown in FIG. 14 have many similarities to the steps of the methods described above, and so some aspects will be described with reference to the method above. Some steps of the method which are the same as those of the method above have been omitted from the flowchart of FIG. 14 for clarity.

The method is performed on reference feature data 10 from individuals in a population. The reference feature data 10 is substantially the same as the feature data 10 described above, but is referred to as reference feature data 10 to clearly distinguish from the subject feature data 15. As above, the population includes a background group of individuals and a target group of individuals at a later stage of the cardiovascular condition than the background group of individuals.

The reference feature data 10 comprises a plurality of features for each individual including a plurality of cardiovascular image features. Although not shown in FIG. 14, the method further comprises a step S210 of determining the cardiovascular image features from images 70 from each of the respective individuals. The disclosure relating to FIG. 2 above applied equally to this method. It is not essential that the method comprise the step S210, and the reference feature data 10 could be received from the output of a separate method or system that produces the reference feature data 10.

The method comprises a step S10 of pre-processing the reference feature data to obtain processed reference feature data 20. This step is not shown in FIG. 14, which shows the processed reference feature data 20 directly. The disclosure above relating to FIG. 3 and the step S10 of pre-processing the feature data applies equally in this method, including that the step S10 of pre-processing the reference feature data is not essential, and the method may use the reference feature data 10 directly.

Although omitted from FIG. 14, the method comprises steps as described above in relation to FIG. 1 for calculating the score 50 representative of a progression of a cardiovascular condition, which may also be referred to as a disease progression score or disease score. The method calculates reference scores 50 comprising scores 50 for each individual in the population.

More specifically, the method comprises applying S20 contrastive principal component analysis between the reference feature data 10 from the background group of individuals and reference feature data 10 from the target group of individuals to obtain a transformation 30 into a reduced representation space. The method then comprises applying S30 the transformation 30 to the reference feature data 10 from the population of individuals to determine a position of each individual of the population in the reduced representation space. The method comprises calculating S35 a matrix of distances among the positions of the individuals of the population in the reduced representation space. This step is not essential, and may be omitted in some cases. The method comprises determining S40 trajectories 40 in the reduced representation space between the target group and the background group by connecting the positions of the individuals of the population in the reduced representation space. The determining S40 of the trajectories is performed on the basis of the matrix of distances where the step S35 is performed. The method comprises for each individual of the population, calculating a reference score 50 representative of the progression of the cardiovascular condition as a distance along the one of the trajectories 40 on which the position of the individual lies.

Following these steps, the method will have calculated reference scores 50 for the individuals in the population using the reference feature data 10 in the same way as described above. All of the disclosure above in relation to these steps applies equally to the steps when performed as part of this alternative method.

As shown in FIG. 14, the method comprises a step S300 of performing fitting between the reference feature data 10 and the reference scores 50 to derive a model 90 for calculating a score representative of the progression of the cardiovascular condition.

Any suitable technique may be used for the fitting used to derive the model 90. For example, the fitting may comprise training a machine learning algorithm such as a neural network. In some embodiments, such as that shown in FIG. 15, the step S300 of performing fitting comprises a step S304 of performing regression analysis, for example linear regression.

The model 90 uses a subset of one or more of the plurality of features to calculate the score. In this context, subset is used in the mathematical sense and includes the possibility that all of the plurality of features are used. However, the subset preferably comprises fewer than all of the plurality of features. For example, the subset may comprise at most 10 features, sometimes at most 5 features, sometimes at most 3 features. While the subset may comprise a single one of the plurality of features, typically it will include plural features.

The subset may be predetermined. Alternatively, as shown in FIG. 15 the fitting may comprise a step S302 of selecting the subset of one or more of the plurality of features. The selection may select a predetermined number of features to be included in the subset, or the number of features in the subset may be determined as part of the selection.

The subset may be selected based on an accuracy of the model 90 using the subset of features. For example, the step S300 may comprise performing fitting between the reference feature data 10 and the reference scores 50 using multiple different subsets of features, and selecting the subset of features to use in the model 90 based on which subset provides the best fit between the reference feature data 10 and the reference scores 50. The step S302 of selecting the subset may comprise using stepwise regression analysis. This method allows for automatically testing different combinations of features to obtain a subset having higher accuracy, and provides a convenient technique for automating part of the step S302 of selecting the subset. The subset may also be selected based on a strength of association between the features and the reference scores 50. The strength of association could be evaluated by any suitable technique, for example by calculating a correlation between each feature and the reference scores 50.

The subset may also be selected based on an ease of obtaining subject feature data 15 comprising data on the subset of features. Some features may be more available or easier to measure than others. For example, if features can be measured using more readily available equipment or do not require specially trained personnel to measure the feature. This may mean that some features are preferred due to the case with which they can be measured, even if the overall accuracy of the resulting model 90 is reduced. The subset may be selected based on a type of the features. The type may be a method by which the features are measured (such as x-ray, echocardiogram, magnetic resonance imaging, blood testing etc.).

The criteria for selecting the subset of features may be combined. For example, an initial selection may be performed based on ease of obtaining data to obtain an initial subset of features smaller than the overall plurality of features. A further selection may then be performed to choose the subset from the initial subset based on which features in the initial subset provide the highest accuracy.

The method comprises a step S310 of applying the model 90 to subject feature data 15 from the test subject to obtain the subject score 55. The subject feature data 15 comprises data on the subset of features for the test subject. Although not shown, the method comprises a step S110 of pre-processing the subject feature data 15 to obtain processed subject feature data 25, and the step 310 in FIG. 14 is performed on the processed subject feature data 25. The step S110 of pre-processing is substantially the same as described above for the methods of FIG. 1. As for FIG. 1, the step S110 of pre-processing may be omitted in some embodiments, and the step S310 may use the subject feature data 15 directly.

The step S310 of applying the model 90 comprises whatever process is appropriate for the type of model 90 used. For example, it may comprise substituting into the appropriate equations the values in the subject feature data 15 for each of the subset of features used by the model 90.

As described above for the method of FIG. 1, the method may be carried out by a system 300 for calculating a subject score 55 representative of a progression of a cardiovascular condition for a test subject. The system 300 comprises a processor configured to carry out the steps of the method. The steps of the method shown in FIG. 14 therefore also represent functional units of the system, which may be, for example, programming functions or dedicated integrated circuits.

The method of FIG. 14 described above includes the steps related to calculating the reference scores 50 from the reference feature data 10 that are discussed in relation to FIG. 1. However, further alternative methods are also provided that make use of previously-calculated reference scores 50. This has the advantage of reducing the computational complexity of the operations that need to be performed at the point of calculating the subject score 55 for a specific test subject.

The first of these further alternative methods is a method of calculating a subject score 55 representative of a progression of a cardiovascular condition for a test subject, wherein the method is performed on reference feature data 10 and reference scores 50 representative of the progression of the cardiovascular condition.

As above, the reference feature data 10 is from individuals in a population including a background group of individuals and a target group of individuals at a later stage of the cardiovascular condition than the background group of individuals. The reference feature data 10 comprises a plurality of features for each individual including a plurality of cardiovascular image features.

The reference scores 50 are obtained by the steps described above of applying S20 contrastive principal component analysis to obtain a transformation 30, applying S30 the transformation 30 to the reference feature data 10 to determine a position of each individual in the reduced representation space, determining $40 trajectories 40 in the reduced representation space, and, for each individual of the population, calculating S50 the reference score 50.

The first alternative method then comprises the step S300 of performing fitting, and the step S310 of applying the model 90 as described above. The method may be carried out by a system comprising a processor configured to carry out the steps of the method.

The second further alternative method is a method of determining a subject score 55 representative of a progression of a cardiovascular condition for a test subject, wherein the method uses a model 90 for calculating a score representative of the progression of the cardiovascular condition. In this case, the model 90 is entirely predetermined, and so the method does not require the reference feature data 10 or the reference scores 50 as inputs. only the model 90 itself.

As discussed above, the model 90 uses a set of one or more features to calculate the score, and the model 90 is derived using reference feature data 10 and reference scores 50. The set of one or more features is a subset of the plurality of features included in the reference feature data 10. The reference scores 50 are calculated as discussed above, and the model 90 is derived by the step S300 of performing fitting between the reference feature data 10 and the reference scores 50.

In this case, the method comprises only the step S310 of applying the model 90 for calculating a score representative of the progression of the cardiovascular condition to subject feature data 15 from the test subject to obtain the subject score 55, the subject feature data comprising data on the subset of features for the test subject.

### Results

Several models derived using the method of FIG. 14 were tested to demonstrate the validity of the alternative method for deriving the disease progression score. In the results discussed above, 68 different features were considered as possible factors affecting the score. In these results for the alternative method, 25 of those features were considered that could be obtained from echocardiogram scans or biochemical measurements. These 25 are shown in Table 6, categorised as echocardiography features and biochemistry features.

TABLE 6
Features considered for the alternative method

| | |
|---|---|
| Echocardiography | Biochemistry |

| | | | |
|---|---|---|---|
| 1. | lvef_bp | 1. | HDL |
| 2. | lvedv_bp | 2. | LDL |
| 3. | lvesv_bp | 3. | Triglyceride |
| 4. | lav_bp | 4. | Glucose |
| 5. | ea | 5. | Cholesterol |
| 6. | tapse | 6. | CRP |
| 7. | lat_e | 7. | Insulin |
| 8. | med_e | 8. | HOMA_S |
| 9. | rv_s | 9. | HOMA_IR |
| 10. | lvgls | 10. | HOMA_B |
| 11. | avr_e | | |
| 12. | lvsv_bp | | |
| 13. | lvco | | |
| 14. | lvrwt | | |
| 15. | lvmass | | |

In a first experiment, the subset of features was chosen to be a single, commonly-used echocardiography feature (LVMass). The results are shown in Tables 7 to 10.

TABLE 7
Results of experiment 1 model using
a single echocardiogram feature

Outcome
Disease progression score

| Predictors | β | P-value |
|---|---|---|
| Model 1: LV mass | −0.001 | <0.0001 |

TABLE 8
Residuals for model shown in Table 7

| Min | 1Q | Median | 3Q | Max |
|---|---|---|---|---|
| −0.87545 | −0.23950 | 0.01035 | 0.21580 | 1.05000 |

TABLE 9
Coefficients for model of Table 7

| | Estimate | Std. Error | t value | Pr(>\|t\|) |
|---|---|---|---|---|
| (Intercept) | 1.2567324 | 0.0934247 | 13.452 | <2e−16 |
| lvmass_bv | −0.0009176 | 0.0007042 | −4.143 | 4.75e−05 |

TABLE 10
Other performance values for model of Table 7

| | |
|---|---|
| Multiple R-squared | 0.06701 |
| Adjusted R-squared | 0.06311 |
| F-statistic | 17.17 on 1 and 239 DF |
| p-value | 4.755e−05 |

In a second experiment, an initial subset was chosen of all 15 common echocardiography features from Table 6. Multi-variable stepwise regression was then performed to select the subset from the initial subset, based on those features that had a statistically-significant association with the reference scores. Three subsets, including one, two, and three features respectively, were evaluated as shown in Tables 11 and 12.

TABLE 11
Features in the subsets of the three models of experiment 2

| No. of Features | Features included |
|---|---|
| 1 | Med_e |
| 2 | Med_e, LV Mass |
| 3 | Med_e, LV Mass, LV RWT |

TABLE 12
Performance statistics for three models of experiment 2

| No. of | | | | | | |
|---|---|---|---|---|---|---|
| Features | RMSE | Rsquared | MAE | RMSESD | RsquaredSD | MAESD |
| 1 | 0.3482965 | 0.04745983 | 0.2742698 | 0.03320647 | 0.03908216 | 0.01943458 |
| 2 | 0.3397534 | 0.08515544 | 0.2678086 | 0.02653523 | 0.05532711 | 0.01616396 |
| 3 | 0.3371386 | 0.10248356 | 0.2647367 | 0.02487279 | 0.06170246 | 0.02144972 |

The model with 3 features had the lowest RMSE, and so was selected as the best combination. Its results are shown in Table 13.

TABLE 13
Results of experiment 2 model using three echocardiogram features

| | |
|---|---|
| | Outcome |
| Predictors | Disease progression score |

| Model 2 - Echo | β | P-value |
|---|---|---|
| Medial E′ | 0.02 | <0.0001 |
| LV RWT | −0.35 | |
| LV Mass | −0.001 | |

In a third experiment, an initial subset was chosen of all 10 common biochemistry features from Table 6. Multi-variable stepwise regression was then performed to select the subset from the initial subset, based on those features that had a statistically-significant association with the reference scores. Two subsets, including one and five features respectively, were evaluated as shown in Table 14 and 15.

TABLE 14
Features in the subsets of the two models of experiment 3

| No. of Features | Features included |
|---|---|
| 1 | HDL |
| 5 | HDL, Glucose, HOMA S, |
| | HOMA IR, HOMA B |

TABLE 15
Performance statistics for two models of experiment 3

| No. of | | | | | | |
|---|---|---|---|---|---|---|
| Features | RMSE | Rsquared | MAE | RMSESD | RsquaredSD | MAESD |
| 1 | 0.3438128 | 0.06140980 | 0.2743847 | 0.04042364 | 0.06128773 | 0.02718822 |
| 5 | 0.3437988 | 0.06503627 | 0.2783139 | 0.03612128 | 0.07257855 | 0.02303415 |

The model with 5 features had the lowest RMSE, and so was selected as the best combination. Its results are shown in Table 16.

TABLE 16
Results of experiment 3 model using five biochemistry features

| | |
|---|---|
| | Outcome |
| Predictors | Disease progression score |

| Model 3 - Biochemistry | β | P-value |
|---|---|---|
| HDL | 0.11 | 0.001 |
| Glucose | −0.15 | |
| HOMA S | 0.0005 | |
| HOMA IR | 0.2 | |
| HOMA B | −0.002 | |

In a fourth experiment, the initial subset was chosen to include all 25 common echocardiogram and biochemistry features from Table 6. Multi-variable stepwise regression was then performed to select the subset from the initial subset, based on those features that had a statistically-significant association with the reference scores. Two subsets, including three and five features respectively, were evaluated as shown in Tables 17 and 18.

TABLE 17
Features in the subsets of the two models of experiment 4

| No. of Features | Features included |
|---|---|
| 3 | Lat_e, lvrwt, HDL |
| 5 | Lat_e, lvrwt, HDL, avr_e, |
| | lvsv_bp |

TABLE 18
Performance statistics for two models of experiment 4

| No. of | | | | | | |
|---|---|---|---|---|---|---|
| Features | RMSE | Rsquared | MAE | RMSESD | RsquaredSD | MAESD |
| 3 | 0.3523436 | 0.07465202 | 0.2874343 | 0.04713272 | 0.10180724 | 0.02905421 |
| 5 | 0.3407332 | 0.08531136 | 0.2718430 | 0.04388987 | 0.07612267 | 0.02942189 |

The model with 5 features had the lowest RMSE, and so was selected as the best combination. Its results are shown in Table 19.

TABLE 16
Results of experiment 4 model using five
echocardiogram and biochemistry features

| | |
|---|---|
| Predictors | Outcome |
| Model 4 - Echo & | Disease progression score |

| biochemistry | β | P-value |
|---|---|---|
| Lateral E′ | −0.03 | <0.0001 |
| Average E′ | 0.04 | |
| LV SV | −0.002 | |
| LV RWT | −0.5 | |
| HDL | 0.1 | |

All of the simple models tested above display a p-value of less than 0.05, indicating a statistically significant result.

### CLAUSES

Aspects of the invention may also be described by the following numbered clause, which correspond to claims of a priority application. These are not the claims of the application, which follow under the heading CLAIMS below.
1. A method of calculating a score representative of a progression of a cardiovascular condition, wherein the method is performed on feature data from individuals in a population including a background group of individuals and a target group of individuals at a later stage of the cardiovascular condition than the background group of individuals, the feature data comprising a plurality of features for each individual including a plurality of cardiovascular image features, and the method comprises: applying contrastive principal component analysis between feature data from the background group of individuals and feature data from the target group of individuals to obtain a transformation into a reduced representation space; applying the transformation to the feature data from the population of individuals to determine a position of each individual of the population in the reduced representation space; determining trajectories in the reduced representation space between the target group and the background group by connecting the positions of the individuals of the population in the reduced representation space; and for one or more of the individuals of the population, calculating the score as a distance along the one of the trajectories on which the position of the individual lies.
2. A method according to clause 1, further comprising calculating a contribution of each of the plurality of features to the transformation, and determining a plurality of the features having the highest contributions to the transformation.
3. A method according to clause 1 or 2, wherein the population further includes a reference group of individuals at a stage of the cardiovascular condition intermediate the background group of individuals and the target group of individuals.
4. A method according to any one of the preceding clauses, wherein the population of individuals further includes at least one test subject at an unknown stage of the cardiovascular condition, and the one or more individuals for whom the score is calculated comprises the at least one test subject.
5. A method according to any one of the preceding clauses, further comprising: calculating a matrix of distances among the positions of the individuals of the population in the reduced representation space, the step of determining trajectories being performed on the basis of the matrix of distances.
6. A method according to clause 5, wherein the step of determining trajectories comprises: determining a minimum spanning tree among the positions of the population in the reduced representation space based on the matrix of distances; and defining the trajectories as paths within the minimum spanning tree.
7. A method according to clause 5 or 6, wherein the distances are Euclidean distances.
8. A method according to any one of clauses 5 to 7, wherein the step of determining trajectories further comprises identifying one or more subtrajectories representing paths in the reduced representation space based on the matrix of distances, each subtrajectory comprising a plurality of the trajectories, and assigning each individual of the population to one or more of the subtrajectories.
9. A method according to clause 8, wherein the identifying of the one or more subtrajectories comprises performing spectral clustering over the matrix of distances.
10. A method according to any one of the preceding clauses, wherein the trajectories connect to a reference point and the distance along the one of the trajectories on which the position of the individual lies is a distance between the position of the individual and the reference point.
11. A method according to clause 10, wherein the reference point is an average position in the reduced representation space of individuals in the background group.
12. A method of analysing feature data about a cardiovascular condition wherein the method is performed on feature data from individuals in a population including a background group of individuals and a target group of individuals at a later stage of the cardiovascular condition than the background group of individuals, the feature data comprising a plurality of features for each individual including a plurality of cardiovascular image features, and the method comprises: applying contrastive principal component analysis between feature data from the background group of individuals and feature data from the target group of individuals to obtain a transformation into a reduced representation space; calculating a contribution of each of the plurality of features to the transformation; and determining a plurality of the features having the highest contributions to the transformation.
13. A method according to clause 12, further comprising: applying the transformation to the feature data from the population of individuals to determine a position of each individual of the population in the reduced representation space; determining trajectories in the reduced representation space between the target group and the background group by connecting the positions of the individuals of the population in the reduced representation space; and for one or more of the individuals of the population, calculating the score as a distance along the one of the trajectories on which the position of the individual lies.
14. A method according to clause 2, 12, or 13, wherein: calculating the contribution comprises, for one or more principal components from the contrastive principal component analysis, calculating a product of an eigenvalue of the principal component with a loading of the feature for the principal component; and the contribution for the feature comprises a sum of the products.
15. A method according to clause 14, wherein the one or more principal components comprises principal components having an eigenvalue above a predetermined value.
16. A method according to clause 14 or 15, wherein the product is normalised by a sum for the principal component of the loadings of the plurality of features.
17. A method according to any one of the proceeding clauses, further comprising a step of pre-processing the feature data to obtain processed feature data, wherein the steps of applying contrastive principal component analysis and applying the transformation are performed using the processed feature data.
18. A method according to clause 16, wherein pre-processing the feature data comprises adjusting the feature data to account for one or more confounding factors.
19. A method according to clause 18, wherein the confounding factors comprise one or more of a sex of each of the individuals, an age of each of the individuals, a condition under which the feature data was measured, and a medication regime of each of the individuals.
20. A method according to any one of clauses 17 to 19, wherein pre-processing the feature data comprises imputing missing values for one or more of the features for one or more of the individuals.
21. A method according to any one of clauses 17 to 20, wherein pre-processing the feature data comprises selecting a subset of the features based on a comparison for each feature of a local variance of the feature with a global variance of the feature.
22. A method according to any one of the preceding clauses, wherein the step of applying contrastive principal component analysis comprises applying a contrast parameter to the feature data from the background group.
23. A method according to clause 22, wherein the step of applying contrastive principal component analysis comprises applying the contrastive principal component analysis a plurality of times using different values of the contrast parameter to obtain a plurality of different transformations, and selecting one of the plurality of transformations, wherein the step of applying the transformation uses the selected transformation.
24. A method according to clause 23, wherein selecting one of the plurality of transformations comprises automatically selecting one of the plurality of transformations.
25. A method according to clause 24, wherein automatically selecting one of the plurality of transformations comprises: for each of the plurality of transformations: determining positions of each of the individuals of the population in a reduced representation space using the transformation; assigning each position to one of a plurality of clusters in the reduced representation space; and calculating a clustering parameter using the positions, the clustering parameter comparing a dispersion within each of the clusters to a reference distribution; selecting a transformation from the plurality of transformations based on the clustering parameter.
26. A method according to any preceding clause, wherein applying contrastive principal component analysis comprises applying kernel contrastive principal component analysis, such that the transformation into the reduced representation space is non-linear.
27. A method of determining a subject score representative of a progression of a cardiovascular condition for a test subject comprising: determining a position of the test subject in a reduced representation space by applying a transformation into the reduced representation space obtained using the method of any one of the preceding clauses to subject feature data from the test subject, the subject feature data comprising data on a plurality of features for the test subject including a plurality of cardiovascular image features; determining a position of the test subject on one of a plurality of trajectories in the reduced representation space determined using the method of clause 1 or any clause dependent thereon; and calculating the subject score using a position along the one of the trajectories on which the position of the subject lies.
28. A method according to clause 1, clause 27, or any clause dependent thereon, wherein the plurality of features is a plurality of features having the highest contributions to the transformation determined using a method according to clause 12 or any clause dependent thereon.
29. A method according to any one of the preceding clauses, wherein the plurality of cardiovascular image features are determined from echocardiogram images.
30. A method according to any one of the preceding clauses, wherein the plurality of cardiovascular image features are determined from cardiac images.
31. A method according to any one of the preceding clauses, wherein the method further comprises a step of determining the cardiovascular image features from images from each of the respective individuals.
32. A method according to any one of the preceding clauses, wherein the feature data further comprises clinical data about each of the respective individuals, the clinical data comprising one or more of: an age of the individual, a sex of the individual, an ethnicity of the individual, a height of the individual, a weight of the individual, and a medication regime of the individual.
33. A method according to any one of the preceding clauses, wherein the cardiovascular condition is hypertension, cardiac disease, or diastolic dysfunction.
34. A computer program comprising instructions which, when the program is executed by a computer, cause the computer to carry out the method of any of clauses 1 to 33.
35. A system for calculating a score representative of a progression of a cardiovascular condition, the system comprising a processor configured to: receive feature data from individuals in a population including a background group of individuals and a target group of individuals at a later stage of the cardiovascular condition than the background group of individuals, the feature data comprising a plurality of features for each individual including a plurality of cardiovascular image features; apply contrastive principal component analysis between feature data from the background group of individuals and feature data from the target group of individuals to obtain a transformation into a reduced representation space; apply the transformation to the feature data from the population of individuals to determine a position of each individual of the population in the reduced representation space; determine trajectories in the reduced representation space between the target group and the background group by connecting the positions of the individuals of the population in the reduced representation space; and for one or more of the individuals of the population, calculate the score as a distance along the one of the trajectories on which the position of the individual lies.
36. A system for analysing feature data about a cardiovascular condition comprising a processor configured to: receive feature data from individuals in a population including a background group of individuals and a target group of individuals at a later stage of the cardiovascular condition than the background group of individuals, the feature data comprising a plurality of features for each individual including a plurality of cardiovascular image features; apply contrastive principal component analysis between feature data from the background group of individuals and feature data from the target group of individuals to obtain a transformation into a reduced representation space; calculate a contribution of each of the plurality of features to the transformation; and determine a plurality of the features having the highest contributions to the transformation.
37. A system for determining a subject score representative of a progression of a cardiovascular condition for a test subject comprising a processor configured to: determine a position of the subject in a reduced representation space by applying a transformation into the reduced representation space obtained using the method of any one of the preceding clauses to subject feature data from the test subject, the subject feature data comprising data on a plurality of features for the test subject including a plurality of cardiovascular image features; determine a position of the subject on one of a plurality of trajectories in the reduced representation space determined using the method of clause 1 or any clause dependent thereon; and calculate the subject score using a position along the one of the trajectories on which the position of the subject lies.
38. A method of calculating a subject score representative of a progression of a cardiovascular condition for a test subject, wherein the method is performed on reference feature data from individuals in a population including a background group of individuals and a target group of individuals at a later stage of the cardiovascular condition than the background group of individuals, the reference feature data comprising a plurality of features for each individual including a plurality of cardiovascular image features, and the method comprises: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; for each individual of the population, calculating a reference score representative of the progression of the cardiovascular condition as a distance along the one of the trajectories on which the position of the individual lies; performing fitting between the reference feature data and the reference scores to derive a model for calculating a score representative of the progression of the cardiovascular condition, wherein the model uses a subset of one or more of the plurality of features to calculate the score; and applying the model to subject feature data from the test subject to obtain the subject score, the subject feature data comprising data on the subset of features for the test subject.
39. A method according to clause 38, wherein the fitting further comprises selecting the subset of one or more of the plurality of features, optionally wherein the subset comprises fewer than all of the plurality of features.
40. A method according to clause 39, wherein the subset is selected based on an accuracy of the model using the subset of features.
41. A method according to clause 39 or 40, wherein the subset is selected based on an ease of obtaining subject feature data comprising data on the subset of features.
42. A method according to any one of clauses 38 to 41, wherein the fitting comprises regression analysis.
43. A method according to clause 42, wherein the regression analysis comprises linear regression.
44. A method according to clause 39 or any preceding clause dependent thereon, wherein selecting the subset comprises using stepwise regression analysis.
45. A method according to any one of the clauses 38 to 44, wherein the population further includes a reference group of individuals at a stage of the cardiovascular condition intermediate the background group of individuals and the target group of individuals.
46. A method according to any one of the clauses 38 to 45, further comprising: calculating a matrix of distances among the positions of the individuals of the population in the reduced representation space, the step of determining trajectories being performed on the basis of the matrix of distances.
47. A method according to clause 46, wherein the step of determining trajectories comprises: determining a minimum spanning tree among the positions of the population in the reduced representation space based on the matrix of distances; and defining the trajectories as paths within the minimum spanning tree.
48. A method according to clause 46 or 47, wherein the distances are Euclidean distances.
49. A method according to any one of clauses 38 to 48, wherein the trajectories connect to a reference point and the distance along the one of the trajectories on which the position of the individual lies is a distance between the position of the individual and the reference point.
50. A method according to clause 49, wherein the reference point is an average position in the reduced representation space of individuals in the background group.
51. A method according to any one of clauses 38 to 50, further comprising a step of pre-processing the feature data to obtain processed feature data, wherein the steps of applying contrastive principal component analysis and applying the transformation are performed using the processed feature data.
52. A method according to clause 51, wherein pre-processing the feature data comprises adjusting the feature data to account for one or more confounding factors.
53. A method according to clause 52, wherein the confounding factors comprise one or more of a sex of each of the individuals, an age of each of the individuals, a condition under which the feature data was measured, and a medication regime of each of the individuals.
54. A method according to any one of clauses 51 to 53, wherein pre-processing the feature data comprises imputing missing values for one or more of the features for one or more of the individuals.
55. A method according to any one of clauses 51 to 54, wherein pre-processing the feature data comprises selecting a subset of the features based on a comparison for each feature of a local variance of the feature with a global variance of the feature.
56. A method according to any one of clauses 38 to 55, wherein the step of applying contrastive principal component analysis comprises applying a contrast parameter to the feature data from the background group.
57. A method according to clause 56, wherein the step of applying contrastive principal component analysis comprises applying the contrastive principal component analysis a plurality of times using different values of the contrast parameter to obtain a plurality of different transformations, and selecting one of the plurality of transformations, wherein the step of applying the transformation uses the selected transformation.
58. A method according to clause 57, wherein selecting one of the plurality of transformations comprises automatically selecting one of the plurality of transformations.
59. A method according to clause 58, wherein automatically selecting one of the plurality of transformations comprises: for each of the plurality of transformations: determining positions of each of the individuals of the population in a reduced representation space using the transformation; assigning each position to one of a plurality of clusters in the reduced representation space; and calculating a clustering parameter using the positions, the clustering parameter comparing a dispersion within each of the clusters to a reference distribution; selecting a transformation from the plurality of transformations based on the clustering parameter.
60. A method according to any one of the clauses 38 to 59, wherein applying contrastive principal component analysis comprises applying kernel contrastive principal component analysis, such that the transformation into the reduced representation space is non-linear.
61. A method according to any one of clauses 38 to 60, wherein the plurality of cardiovascular image features are determined from echocardiogram images.
62. A method according to any one of clauses 38 to 61, wherein the plurality of cardiovascular image features are determined from cardiac images.
63. A method according to any one of clauses 38 to 62, wherein the method further comprises a step of determining the cardiovascular image features from images from each of the respective individuals.
64. A method according to any one of clauses 38 to 63, wherein the feature data further comprises clinical data about each of the respective individuals, the clinical data comprising one or more of: an age of the individual, a sex of the individual, an ethnicity of the individual, a height of the individual, a weight of the individual, and a medication regime of the individual.
65. A method according to any one of clauses 38 to 64, wherein the cardiovascular condition is hypertension, cardiac disease, or diastolic dysfunction.
66. A method of calculating a subject score representative of a progression of a cardiovascular condition for a test subject, wherein the method is performed on reference feature data and reference scores representative of the progression of the cardiovascular condition; the reference feature data is from individuals in a population including a background group of individuals and a target group of individuals at a later stage of the cardiovascular condition than the background group of individuals, the reference feature data comprising a plurality of features for each individual including a plurality of cardiovascular image features; the reference scores are obtained by: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; and for each individual of the population, calculating the reference score as a distance along the one of the trajectories on which the position of the individual lies; and the method comprises: performing fitting between the reference feature data and the reference scores to derive a model for calculating a score representative of the progression of the cardiovascular condition, wherein the model uses a subset of one or more of the plurality of features to calculate the score; and applying the model to subject feature data from the test subject to obtain the subject score, the subject feature data comprising data on the subset of features for the test subject.
67. A method of determining a subject score representative of a progression of a cardiovascular condition for a test subject, wherein: the method uses a model for calculating a score representative of the progression of the cardiovascular condition; the model uses a set of one or more features to calculate the score; the model is derived using reference feature data from individuals in a population including a background group of individuals and a target group of individuals at a later stage of the cardiovascular condition than the background group of individuals, the reference feature data comprising a plurality of features for each individual including a plurality of cardiovascular image features, and the model is derived by: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; for each individual of the population, calculating a reference score representative of the progression of the cardiovascular condition as a distance along the one of the trajectories on which the position of the individual lies; performing fitting between the reference feature data and the reference scores to derive the model for calculating a score representative of the progression of the cardiovascular condition using the set of one or more features, wherein the set of one or more features is a subset of the plurality of features; the method comprises: applying the model for calculating a score representative of the progression of the cardiovascular condition to subject feature data from the test subject to obtain the subject score, the subject feature data comprising data on the set of features for the test subject.
68. A computer program comprising instructions which, when the program is executed by a computer, cause the computer to carry out the method of any of clauses 38 to 65.
69. A system for calculating a subject score representative of a progression of a cardiovascular condition for a test subject, the system comprising a processor configured to: receive reference feature data from individuals in a population including a background group of individuals and a target group of individuals at a later stage of the cardiovascular condition than the background group of individuals, the reference feature data comprising a plurality of features for each individual including a plurality of cardiovascular image features; apply contrastive principal component analysis between reference feature data from the background group of individuals and reference feature data from the target group of individuals to obtain a transformation into a reduced representation space; apply the transformation to the reference feature data from the population of individuals to determine a position of each individual of the population in the reduced representation space; determine trajectories in the reduced representation space between the target group and the background group by connecting the positions of the individuals of the population in the reduced representation space; and for each individual of the population, calculate a reference score representative of the progression of the cardiovascular condition as a distance along the one of the trajectories on which the position of the individual lies; perform fitting between the reference feature data and the reference scores to derive a model for calculating the score representative of the progression of the cardiovascular condition, wherein the model uses a subset of one or more of the plurality of features to calculate the score; and apply the model to subject feature data from the test subject to obtain the subject score, the subject feature data comprising data on the subset of features for the test subject.
70. A system for calculating a subject score representative of a progression of a cardiovascular condition for a test subject, the system comprising a processor configured to: receive reference feature data and reference scores representative of the progression of the cardiovascular condition, wherein: the reference feature data is from individuals in a population including a background group of individuals and a target group of individuals at a later stage of the cardiovascular condition than the background group of individuals, the reference feature data comprising a plurality of features for each individual including a plurality of cardiovascular image features; and the reference scores are obtained by: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; and for each individual of the population, calculating a reference score representative of the progression of the cardiovascular condition as a distance along the one of the trajectories on which the position of the individual lies; wherein the processor is further configured to: perform fitting between the reference feature data and the reference scores to derive a model for calculating the score representative of the progression of the cardiovascular condition, wherein the model uses a subset of one or more of the plurality of features to calculate the score; and apply the model to subject feature data from the test subject to obtain the subject score, the subject feature data comprising data on the subset of features for the test subject.
71. A system for calculating a subject score representative of a progression of a cardiovascular condition for a test subject, the system comprising a processor configured to apply a model for calculating a score representative of the progression of the cardiovascular condition, wherein: the model uses a set of one or more features to calculate the score; the model is derived using reference feature data is from individuals in a population including a background group of individuals and a target group of individuals at a later stage of the cardiovascular condition than the background group of individuals, the reference feature data comprising a plurality of features for each individual including a plurality of cardiovascular image features; and the model is derived by: applying contrastive principal component analysis between reference feature data from the background group of individuals and reference feature data from the target group of individuals to obtain a transformation into a reduced representation space; applying the transformation to the reference feature data from the population of individuals to determine a position of each individual of the population in the reduced representation space; determining trajectories in the reduced representation space between the target group and the background group by connecting the positions of the individuals of the population in the reduced representation space; and for each individual of the population, calculating a reference score representative of the progression of the cardiovascular condition as a distance along the one of the trajectories on which the position of the individual lies; and performing fitting between the reference feature data and the reference scores to derive the model for calculating the score representative of the progression of the cardiovascular condition using the set of one or more features, wherein the set of one or more features is a subset of the plurality of features; wherein the processor is configured to: apply the model for calculating a score representative of the progression of the cardiovascular condition to subject feature data from the test subject to obtain the subject score, the subject feature data comprising data on the set of features for the test subject.

### REFERENCES

1. Lewington S, Clarke R, Qizilbash N, et al. Age-specific relevance of usual blood pressure to vascular mortality: a meta-analysis of individual data for one million adults in 61 prospective studies. Lancet 2002; 360: 1903-1913. 2002 Dec. 21. DOI: 10.1016/s0140-6736(02)11911-8.
2. Bergman E M, Henriksson Km Fau-Åsberg S, Åsberg S Fau-Farahmand B, et al. National registry-based case-control study: comorbidity and stroke in young adults.
3. Deedwania P C. The Progression From Hypertension to Heart Failure. American Journal of Hypertension 1997; 10: 280S-288S. DOI: 10.1016/S0895-7061(97)00335-X.
4. Drazner M H. The Progression of Hypertensive Heart Disease. Circulation 2011; 123: 327-334. DOI: 10.1161/CIRCULATIONAHA.108.845792.
5. Mancia G, Fagard R, Narkiewicz K, et al. 2013 ESH/ESC Guidelines for the management of arterial hypertension: the Task Force for the management of arterial hypertension of the European Society of Hypertension (ESH) and of the European Society of Cardiology (ESC). Journal of hypertension 2013; 31: 1281-1357. 2013 Jul. 3. DOI: 10.1097/01.hjh.0000431740.32696.cc.
6. Zanchetti A, Dominiczak A, Coca A, et al. 2018 ESC/ESH Guidelines for the management of arterial hypertension. European Heart Journal 2018; 39: 3021-3104. DOI: 10.1093/eurheartj/ehy339%J European Heart Journal.
7. Whelton P K, Carey R M, Aronow W S, et al. 2017
   ACC/AHA/AAPA/ABC/ACPM/AGS/APhA/ASH/ASPC/NMA/PCNA Guideline for the Prevention, Detection, Evaluation, and Management of High Blood Pressure in Adults: Executive Summary: A Report of the American College of Cardiology/American Heart Association Task Force on Clinical Practice Guidelines. Circulation 2018; 138: e426-e483. 2018 Oct. 26. DOI: 10.1161/cir.0000000000000597.
8. Bergman E M, Henriksson K M, Asberg S, et al. National registry-based case-control study: comorbidity and stroke in young adults. Acta neurologica Scandinavica 2015; 131: 394-399. 2015 Feb. 17. DOI: 10.1111/ane.12265.
9. Lewington S, Clarke R, Qizilbash N, et al. Age-specific relevance of usual blood pressure to vascular mortality: a meta-analysis of individual data for one million adults in 61 prospective studies. Lancet 2002; 360: 1903-1913. 2002 Dec. 21.
10. Cameli M, Lisi M, Focardi M, et al. Left Atrial Deformation Analysis by Speckle Tracking Echocardiography for Prediction of Cardiovascular Outcomes. The American Journal of Cardiology 2012; 110: 264-269. DOI:
   https://doi.org/10.1016/j.amjcard.2012.03.022.
11. Santos A B, Roca G Q, Claggett B, et al. Prognostic Relevance of Left Atrial Dysfunction in Heart Failure With Preserved Ejection Fraction. Circulation Heart failure 2016; 9: e002763. 2016 Apr. 9. DOI: 10.1161/circheartfailure.115.002763.
12. HARRELL Jr. F E, LEE K L and MARK D B. MULTIVARIABLE PROGNOSTIC MODELS: ISSUES IN DEVELOPING MODELS, EVALUATING ASSUMPTIONS AND ADEQUACY, AND MEASURING AND REDUCING ERRORS. Statistics in Medicine 1996; 15: 361-387. DOI: 10.1002/(sici)1097-0258(19960229)15:4<361::Aid-sim168>3.0.Co; 2-4.
13. Andreas Mayr H B, Olaf Gefeller, Matthias Schmid. The Evolution of Boosting Algorithms-From Machine Learning to Statistical Modelling. Methods Inf Med 2014; 53: 419-427.
14. Gupta A and Bar-Joseph Z. Extracting Dynamics from Static Cancer Expression Data. IEEE/ACM Transactions on Computational Biology and Bioinformatics 2008; 5: 172-182. DOI: 10.1109/TCBB.2007.70233.
15. Campbell K R and Yau C. Uncovering pseudotemporal trajectories with covariates from single cell and bulk expression data. Nat Commun 2018; 9: 2442. DOI: doi:10.1038/s41467-018-04696-6.
16. Magwene P M, Lizardi P Fau-Kim J and Kim J. Reconstructing the temporal ordering of biological samples using microarray data. Bioinformatics 2003; 19: 842-850. DOI: 10.1093/bioinformatics/btg081.
17. Iturria-Medina Y, Khan A F, Adewale Q, et al. Blood and brain gene expression trajectories mirror neuropathology and clinical deterioration in neurodegeneration. Brain 2020; 143: 661-673. DOI: 10.1093/brain/awz400.
18. Abid A, Zhang M J, Bagaria V K, et al. Exploring patterns enriched in a dataset with contrastive principal component analysis. Nature Communications 2018; 9: 2134. DOI: 10.1038/s41467-018-04608-8.
19. Street J O, Carroll R J and Ruppert D. A Note on Computing Robust Regression Estimates Via Iteratively Reweighted Least Squares. The American Statistician 1988; 42: 152-154. DOI: 10.2307/2684491.
20. Welch J D, Hartemink, A. J. & Prins, J. F. SLICER: inferring branched, nonlinear cellular trajectories from single cell RNA-seq data. Genome Biol 2016; 17. DOI: 10.1186/s13059-016-0975-3.
21. Iturria-Medina Y, Carbonell F, Assadi A, et al. Integrating molecular, histopathological, neuroimaging and clinical neuroscience data with NeuroPM-box. Communications Biology 2021; 4: 614. DOI: 10.1038/s42003-021-02133-x.
22. Ng A Y, Jordan M I and Weiss Y. On spectral clustering: analysis and an algorithm. Proceedings of the 14th International Conference on Neural Information Processing Systems: Natural and Synthetic. Vancouver, British Columbia, Canada: MIT Press, 2001, p. 849-856.
23. Hespanha J. An Efficient MATLAB Algorithm for Graph Partitioning Technical Report. In: 2004.
24. Miao J and Ben-Israel A. On principal angles between subspaces in Rn. Linear Algebra and its Applications 1992; 171: 81-98. DOI: https://doi.org/10.1016/0024-3795(92)90251-5.
25. Tibshirani R, Walther G and Hastie T. Estimating the number of clusters in a data set via the gap statistic. Journal of the Royal Statistical Society: Series B (Statistical Methodology) 2001; 63: 411-423. DOI: https://doi.org/10.1111/1467-9868.00293.
26. Abdi H and Williams L J. Principal component analysis. WIREs Computational Statistics 2010; 2: 433-459. DOI: https://doi.org/10.1002/wics.101.
27. Williamson W, Lewandowski A J, Forkert N D, et al. Association of Cardiovascular Risk Factors With MRI Indices of Cerebrovascular Structure and Function and White Matter Hyperintensities in Young Adults. JAMA 2018; 320: 665-673. DOI: 10.1001/jama.2018.11498.
28. Williamson W. Huckstep O J, Frangou E, et al. Trial of Exercise to Prevent Hypertension in young Adults (TEPHRA) a randomized controlled trial: study protocol. BMC Cardiovascular Disorders 2018; 18: 208. DOI: 10.1186/s12872-018-0944-8.
29. Harkness A, Ring L, Augustine D X, et al. Normal reference intervals for cardiac dimensions and function for use in echocardiographic practice: a guideline from the British Society of Echocardiography. Echo Res Pract 2020; 7: G1-G18. DOI: 10.1530/ERP-19-0050.
30. Lang R M, Badano L P, Mor-Avi V, et al. Recommendations for Cardiac Chamber Quantification by Echocardiography in Adults: An Update from the American Society of Echocardiography and the European Association of Cardiovascular Imaging. European Heart Journal—Cardiovascular Imaging 2015; 16: 233-271. DOI: 10.1093/ehjci/jev014.
31. Nishimura Ra Fau-Otto C M, Otto Cm Fau-Bonow R O, Bonow Ro Fau-Carabello B A, et al. 2014 AHA/ACC Guideline for the Management of Patients With Valvular Heart Disease: executive summary: a report of the American College of Cardiology/American Heart Association Task Force on Practice Guidelines.
32. Badano L P, Kolias T J, Muraru D, et al. Standardization of left atrial, right ventricular, and right atrial deformation imaging using two-dimensional speckle tracking echocardiography: a consensus document of the EACVI/ASE/Industry Task Force to standardize deformation imaging.
33. Modin D, Biering-Sørensen Sofie R. Mogelvang R, et al. Prognostic Value of Echocardiography in Hypertensive Versus Nonhypertensive Participants From the General Population. Hypertension 2018; 71: 742-751. DOI:

### 10.1161/HYPERTENSIONAHA.117.10674.

34. Freed B H, Daruwalla V, Cheng J Y, et al. Prognostic Utility and Clinical Significance of Cardiac Mechanics in Heart Failure With Preserved Ejection Fraction: Importance of Left Atrial Strain. Circ Cardiovasc Imaging 2016; 9:
   10.1161/CIRCIMAGING.1115.003754 e003754. DOI:

### 10.1161/CIRCIMAGING.115.003754.

35. Mayet J and Hughes A. Cardiac and vascular pathophysiology in hypertension. Heart 2003; 89: 1104-1109. DOI: 10.1136/heart.89.9.1104.
36. Katz D H, Deo R C, Aguilar F G, et al. Phenomapping for the Identification of Hypertensive Patients with the Myocardial Substrate for Heart Failure with Preserved Ejection Fraction. J of Cardiovasc Trans Res 2017; 10: 275-284. DOI: 10.1007/s12265-017-9739-z.
37. Tokodi M, Shrestha S, Bianco C, et al. Interpatient Similarities in Cardiac Function. JACC: Cardiovascular Imaging 2020; 13: 1119. DOI:
   10.1016/j.jcmg.2019.12.018.
38. Shah S J, Katz D H, Selvaraj S, et al. Phenomapping for Novel Classification of Heart Failure With Preserved Ejection Fraction. Circulation 2015; 131: 269-279. DOI: 10.1161/CIRCULATIONAHA.114.010637.
39. Sanchez-Martinez S, Duchateau N, Erdei T, et al. Machine Learning Analysis of Left Ventricular Function to Characterize Heart Failure With Preserved Ejection Fraction. Circulation: Cardiovascular Imaging 2018; 11: e007138. DOI:

### doi: 10.1161/CIRCIMAGING.117.007138.

1. A method of calculating a score representative of a progression of a cardiovascular condition, wherein
the method is performed on feature data from individuals in a population including a background group of individuals and a target group of individuals at a later stage of the cardiovascular condition than the background group of individuals, the feature data comprising a plurality of features for each individual including a plurality of cardiovascular image features, and
the method comprises:
applying contrastive principal component analysis between feature data from the background group of individuals and feature data from the target group of individuals to obtain a transformation into a reduced representation space;
applying the transformation to the feature data from the population of individuals to determine a position of each individual of the population in the reduced representation space;
determining trajectories in the reduced representation space between the target group and the background group by connecting the positions of the individuals of the population in the reduced representation space; and
for one or more of the individuals of the population, calculating the score as a distance along the one of the trajectories on which the position of the individual lies.

2. A method according to claim 1, wherein the population of individuals further includes at least one test subject at an unknown stage of the cardiovascular condition, and the one or more individuals for whom the score is calculated comprises the at least one test subject.

3. A method of calculating a subject score representative of a progression of a cardiovascular condition for a test subject, wherein
the method is performed on reference feature data from individuals in a population including a background group of individuals and a target group of individuals at a later stage of the cardiovascular condition than the background group of individuals, the reference feature data comprising a plurality of features for each individual including a plurality of cardiovascular image features, and
the method comprises:
applying contrastive principal component analysis between reference feature data from the background group of individuals and reference feature data from the target group of individuals to obtain a transformation into a reduced representation space;
applying the transformation to the reference feature data from the population of individuals to determine a position of each individual of the population in the reduced representation space;
determining trajectories in the reduced representation space between the target group and the background group by connecting the positions of the individuals of the population in the reduced representation space;
for each individual of the population, calculating a reference score representative of the progression of the cardiovascular condition as a distance along the one of the trajectories on which the position of the individual lies;
performing fitting between the reference feature data and the reference scores to derive a model for calculating a score representative of the progression of the cardiovascular condition, wherein the model uses a subset of one or more of the plurality of features to calculate the score; and
applying the model to subject feature data from the test subject to obtain the subject score, the subject feature data comprising data on the subset of features for the test subject.

4. A method according to claim 3, wherein the fitting further comprises selecting the subset of one or more of the plurality of features, optionally wherein the subset comprises fewer than all of the plurality of features.

5. A method according to claim 4, wherein the subset is selected based on an accuracy of the model using the subset of features.

6. A method according to claim 4, wherein the subset is selected based on an ease of obtaining subject feature data comprising data on the subset of features.

7. A method according to claim 4, wherein the fitting comprises regression analysis, optionally linear regression.

8. (canceled)

9. A method according to claim 4, wherein selecting the subset comprises using stepwise regression analysis.

10. A method according to claim 1, wherein the population further includes a reference group of individuals at a stage of the cardiovascular condition intermediate the background group of individuals and the target group of individuals.

11. A method according to claim 1, further comprising:
calculating a matrix of distances among the positions of the individuals of the population in the reduced representation space, the step of determining trajectories being performed on the basis of the matrix of distances.

12. A method according to claim 11, wherein the step of determining trajectories comprises:
determining a minimum spanning tree among the positions of the population in the reduced representation space based on the matrix of distances, optionally where the distances an Euclidean distances; and
defining the trajectories as paths within the minimum spanning tree.

13. (canceled)

14. A method according to claim 11, wherein the step of determining trajectories further comprises identifying one or more subtrajectories representing paths in the reduced representation space based on the matrix of distances, each subtrajectory comprising a plurality of the trajectories, and assigning each individual of the population to one or more of the subtrajectories.

15. A method according to claim 14, wherein the identifying of the one or more subtrajectories comprises performing spectral clustering over the matrix of distances.

16. A method according to claim 1, wherein the trajectories connect to a reference point and the distance along the one of the trajectories on which the position of the individual lies is a distance between the position of the individual and the reference point.

17. A method according to claim 16, wherein the reference point is an average position in the reduced representation space of individuals in the background group.

18. A method according to claim 1, further comprising a step of pre-processing the feature data to obtain processed feature data, wherein the steps of applying contrastive principal component analysis and applying the transformation are performed using the processed feature data.

19. A method according to claim 18, wherein pre-processing the feature data comprises adjusting the feature data to account for one or more confounding factors.

20. A method according to claim 19, wherein the confounding factors comprise one or more of a sex of each of the individuals, an age of each of the individuals, a condition under which the feature data was measured, and a medication regime of each of the individuals.

21. A method according to claim 18, wherein pre-processing the feature data comprises imputing missing values for one or more of the features for one or more of the individuals.

22. A method according to claim 18, wherein pre-processing the feature data comprises selecting a subset of the features based on a comparison for each feature of a local variance of the feature with a global variance of the feature.

23. A method according to claim 1, wherein the step of applying contrastive principal component analysis comprises applying a contrast parameter to the feature data from the background group.

24. A method according to claim 23, wherein the step of applying contrastive principal component analysis comprises applying the contrastive principal component analysis a plurality of times using different values of the contrast parameter to obtain a plurality of different transformations, and selecting one of the plurality of transformations, wherein the step of applying the transformation uses the selected transformation.

25. A method according to claim 24, wherein selecting one of the plurality of transformations comprises automatically selecting one of the plurality of transformations.

26. A method according to claim 25, wherein automatically selecting one of the plurality of transformations comprises:
for each of the plurality of transformations: determining positions of each of the individuals of the population in a reduced representation space using the transformation; assigning each position to one of a plurality of clusters in the reduced representation space; and calculating a clustering parameter using the positions, the clustering parameter comparing a dispersion within each of the clusters to a reference distribution;
selecting a transformation from the plurality of transformations based on the clustering parameter.

27. A method according to claim 1, wherein applying contrastive principal component analysis comprises applying kernel contrastive principal component analysis, such that the transformation into the reduced representation space is non-linear.

28. A method according to claim 1, wherein the plurality of cardiovascular image features are determined from echocardiogram images or cardiac images.

29. (canceled)

30. A method according to claim 1, wherein the method further comprises a step of determining the cardiovascular image features from images from each of the respective individuals.

31. A method according to claim 1, wherein the feature data further comprises clinical data about each of the respective individuals, the clinical data comprising one or more of: an age of the individual, a sex of the individual, an ethnicity of the individual, a height of the individual, a weight of the individual, or a medication regime of the individual.

32. A method according to claim 1, wherein the cardiovascular condition is hypertension, cardiac disease, or diastolic dysfunction.

33. A method of determining a subject score representative of a progression of a cardiovascular condition for a test subject comprising:
determining a position of the test subject in a reduced representation space by applying a transformation into the reduced representation space obtained using the method of claim 1 or any preceding claim dependent thereon to subject feature data from the test subject, the subject feature data comprising data on a plurality of features for the test subject including a plurality of cardiovascular image features;
determining a position of the test subject on one of a plurality of trajectories in the reduced representation space determined using the method of claim 1 or any preceding claim dependent thereon; and
calculating the subject score using a position along the one of the trajectories on which the position of the subject lies.

34. A method of calculating a subject score representative of a progression of a cardiovascular condition for a test subject, wherein
the method is performed on reference feature data and reference scores representative of the progression of the cardiovascular condition;
the reference feature data is from individuals in a population including a background group of individuals and a target group of individuals at a later stage of the cardiovascular condition than the background group of individuals, the reference feature data comprising a plurality of features for each individual including a plurality of cardiovascular image features;
the reference scores are obtained by:
applying contrastive principal component analysis between reference feature data from the background group of individuals and reference feature data from the target group of individuals to obtain a transformation into a reduced representation space;
applying the transformation to the reference feature data from the population of individuals to determine a position of each individual of the population in the reduced representation space;
determining trajectories in the reduced representation space between the target group and the background group by connecting the positions of the individuals of the population in the reduced representation space; and
for each individual of the population, calculating the reference score as a distance along the one of the trajectories on which the position of the individual lies; and
the method comprises:
performing fitting between the reference feature data and the reference scores to derive a model for calculating a score representative of the progression of the cardiovascular condition, wherein the model uses a subset of one or more of the plurality of features to calculate the score; and
applying the model to subject feature data from the test subject to obtain the subject score, the subject feature data comprising data on the subset of features for the test subject.

35. A method of determining a subject score representative of a progression of a cardiovascular condition for a test subject, wherein:
the method uses a model for calculating a score representative of the progression of the cardiovascular condition;
the model uses a set of one or more features to calculate the score;
the model is derived using reference feature data from individuals in a population including a background group of individuals and a target group of individuals at a later stage of the cardiovascular condition than the background group of individuals, the reference feature data comprising a plurality of features for each individual including a plurality of cardiovascular image features, and
the model is derived by:
applying contrastive principal component analysis between reference feature data from the background group of individuals and reference feature data from the target group of individuals to obtain a transformation into a reduced representation space;
applying the transformation to the reference feature data from the population of individuals to determine a position of each individual of the population in the reduced representation space;
determining trajectories in the reduced representation space between the target group and the background group by connecting the positions of the individuals of the population in the reduced representation space;
for each individual of the population, calculating a reference score representative of the progression of the cardiovascular condition as a distance along the one of the trajectories on which the position of the individual lies;
performing fitting between the reference feature data and the reference scores to derive the model for calculating a score representative of the progression of the cardiovascular condition using the set of one or more features, wherein the set of one or more features is a subset of the plurality of features;
the method comprises:
applying the model for calculating a score representative of the progression of the cardiovascular condition to subject feature data from the test subject to obtain the subject score, the subject feature data comprising data on the set of features for the test subject.

36. A computer program comprising instructions, or a non-transitory storage medium storing instructions, which, when the instructions are executed by a computer, cause the computer to carry out the method of claim 1.

37. A system for calculating a score representative of a progression of a cardiovascular condition, the system comprising a processor configured to:
receive feature data from individuals in a population including a background group of individuals and a target group of individuals at a later stage of the cardiovascular condition than the background group of individuals, the feature data comprising a plurality of features for each individual including a plurality of cardiovascular image features;
apply contrastive principal component analysis between feature data from the background group of individuals and feature data from the target group of individuals to obtain a transformation into a reduced representation space;
apply the transformation to the feature data from the population of individuals to determine a position of each individual of the population in the reduced representation space;
determine trajectories in the reduced representation space between the target group and the background group by connecting the positions of the individuals of the population in the reduced representation space; and
for one or more of the individuals of the population, calculate the score as a distance along the one of the trajectories on which the position of the individual lies.

38. A system for determining a subject score representative of a progression of a cardiovascular condition for a test subject comprising a processor configured to:
determine a position of the subject in a reduced representation space by applying a transformation into the reduced representation space obtained using the method of claim 1 or any preceding claim dependent thereon to subject feature data from the test subject, the subject feature data comprising data on a plurality of features for the test subject including a plurality of cardiovascular image features;
determine a position of the subject on one of a plurality of trajectories in the reduced representation space determined using the method of claim 1 or any preceding claim dependent thereon; and
calculate the subject score using a position along the one of the trajectories on which the position of the subject lies.

39. A system for calculating a subject score representative of a progression of a cardiovascular condition for a test subject, the system comprising a processor configured to:
receive reference feature data from individuals in a population including a background group of individuals and a target group of individuals at a later stage of the cardiovascular condition than the background group of individuals, the reference feature data comprising a plurality of features for each individual including a plurality of cardiovascular image features;
apply contrastive principal component analysis between reference feature data from the background group of individuals and reference feature data from the target group of individuals to obtain a transformation into a reduced representation space;
apply the transformation to the reference feature data from the population of individuals to determine a position of each individual of the population in the reduced representation space;
determine trajectories in the reduced representation space between the target group and the background group by connecting the positions of the individuals of the population in the reduced representation space; and
for each individual of the population, calculate a reference score representative of the progression of the cardiovascular condition as a distance along the one of the trajectories on which the position of the individual lies;
perform fitting between the reference feature data and the reference scores to derive a model for calculating the score representative of the progression of the cardiovascular condition, wherein the model uses a subset of one or more of the plurality of features to calculate the score; and
apply the model to subject feature data from the test subject to obtain the subject score, the subject feature data comprising data on the subset of features for the test subject.

40. A system for calculating a subject score representative of a progression of a cardiovascular condition for a test subject, the system comprising a processor configured to:
receive reference feature data and reference scores representative of the progression of the cardiovascular condition, wherein:
the reference feature data is from individuals in a population including a background group of individuals and a target group of individuals at a later stage of the cardiovascular condition than the background group of individuals, the reference feature data comprising a plurality of features for each individual including a plurality of cardiovascular image features; and
the reference scores are obtained by:
applying contrastive principal component analysis between reference feature data from the background group of individuals and reference feature data from the target group of individuals to obtain a transformation into a reduced representation space;
applying the transformation to the reference feature data from the population of individuals to determine a position of each individual of the population in the reduced representation space;
determining trajectories in the reduced representation space between the target group and the background group by connecting the positions of the individuals of the population in the reduced representation space; and
for each individual of the population, calculating a reference score representative of the progression of the cardiovascular condition as a distance along the one of the trajectories on which the position of the individual lies;
wherein the processor is further configured to:
perform fitting between the reference feature data and the reference scores to derive a model for calculating the score representative of the progression of the cardiovascular condition, wherein the model uses a subset of one or more of the plurality of features to calculate the score; and
apply the model to subject feature data from the test subject to obtain the subject score, the subject feature data comprising data on the subset of features for the test subject.

41. A system for calculating a subject score representative of a progression of a cardiovascular condition for a test subject, the system comprising a processor configured to apply a model for calculating a score representative of the progression of the cardiovascular condition, wherein:
the model uses a set of one or more features to calculate the score;
the model is derived using reference feature data is from individuals in a population including a background group of individuals and a target group of individuals at a later stage of the cardiovascular condition than the background group of individuals, the reference feature data comprising a plurality of features for each individual including a plurality of cardiovascular image features; and
the model is derived by:
applying contrastive principal component analysis between reference feature data from the background group of individuals and reference feature data from the target group of individuals to obtain a transformation into a reduced representation space;
applying the transformation to the reference feature data from the population of individuals to determine a position of each individual of the population in the reduced representation space;
determining trajectories in the reduced representation space between the target group and the background group by connecting the positions of the individuals of the population in the reduced representation space; and
for each individual of the population, calculating a reference score representative of the progression of the cardiovascular condition as a distance along the one of the trajectories on which the position of the individual lies; and
performing fitting between the reference feature data and the reference scores to derive the model for calculating the score representative of the progression of the cardiovascular condition using the set of one or more features, wherein the set of one or more features is a subset of the plurality of features;
wherein the processor is configured to:
apply the model for calculating a score representative of the progression of the cardiovascular condition to subject feature data from the test subject to obtain the subject score, the subject feature data comprising data on the set of features for the test subject.
